(19) United States
(12) Patent Application Publication
(10) Pub. No.: US 2005/0245472 A1
(43) Pub. Date: Nov. 3, 2005

(54) Engineered rnai adenovirus silencing expression (erase) of dna reair proteins

(75) Inventors: Theodore L. DeWeese, Baltimore, MD (US); Spencer J. Collins, Baltimore, MD (US); William G. Nelson, Towson, MD (US)

Correspondence Address: Peter F Coreless;Edwards & Angell, PO Box 55874, Boston, MA, 02205, US

(73) Assignee: The Johns Hopkins University, Baltimore, MD (US)

(21) Appl. No.: 10/534,010

(22) PCT Filed: Nov. 12, 2003
(86) PCT No.: PCT/US03/36367

§ 371 Date: May 5, 2005

(51) Int. Cl. A61K 48/00; A61K 31/396; A61K 31/137; A61K 31/28; A61K 31/66

(52) U.S. Cl. 514/44; 514/183; 514/109; 514/492; 514/649

(57) ABSTRACT

The present invention relates to compositions and methods for killing cancer cells and treating subjects having cancer comprising administering small inhibitory RNAs (siRNA) specific for DNA repair proteins in conjunction with administration of conventional DNA-damaging agents such as radiation and chemotherapeutic agents.

### RELATED APPLICATIONS

This application claims the benefit of U.S. Provisional Application Ser. No. 60/425,897, filed Nov. 12, 2002. The entire contents of this application is incorporated herein by this reference.

### GOVERNMENT SUPPORT

This work described herein was supported by a grant from the Department of Defense (Grant No. DAMD17-98-1-8475). Therefore, the U.S. Government may have certain rights in the invention.

### BACKGROUND OF THE INVENTION

The development of cancer in mammals is caused by the accumulation of tumorigenic DNA lesions. Highly specialized DNA repair proteins work together with upstream sensors and signalers to protect mammalian cells from such tumorigenic and lethal lesions, which result from DNA damage (for recent reviews, see Jackson, S. P. (2001) Biochem. Soc. Trans. 29:655-661; Hoeijmakers, J. H. (2001) Nature, 411:366-374). Pivotal to these repair pathways are the DNA damage sensors ATM and ATR and DNA-PK_{cs}. The essential role these proteins play in DNA damage and repair is highlighted by the extreme sensitivity to DNA-damaging agents exhibited by cells and animal models defective in and/or lacking ATM, ATR, and DNA-PK_{cs} expression (see-Miller, S. P. et al. (1995) Science, 267:1183-1185; Meyn, M. S. (1995) Cancer Res., 55: 5991-6001; Shiloh, Y. (2001) Curr. Opin. Genet. Dev. 11:71-77). Because DNA-damaging agents, including radiation and chemotherapeutic agents, are also used to treat cancer by inducing lethal DNA damage, targeted inhibition of these kinases is an attractive approach in the development of cancer therapy strategies.

Proteins involved in the detection, signaling, and repair of DNA damage after exposure to cytotoxic agents are attractive targets when considering exogenous modulation of DNA repair capacity as a means to increase cellular sensitivity to ionizing radiation and/or chemotherapeutic agents. As such, controlled targeted inhibition of the DNA damage signaling/repair factors ATM, ATR, and DNA-PKcs combined with localized conformal radiotherapy or systemic delivery of chemotherapeutic drugs would make an attractive adjuvant gene therapy approach for many solid tumors. Accordingly, there is a need in the art for methods which can effectively inhibit these kinases in conjunction with DNA-damaging agents.

### SUMMARY OF THE INVENTION

The present invention is based, at least in part, on the discovery that inhibition of DNA repair protein expression through the use of small inhibitory RNA (siRNA) can augment radiation and chemotherapy-mediated killing of cancer cells by sensitizing the cells to the DNA-damaging action of those therapies. Accordingly, the present invention provides methods for killing cancer cells and methods of treating subjects having cancer, comprising administering siRNAs directed to DNA repair proteins (e.g., ATM and ATR and DNA-PK_{cs}) in combination with one or more DNA-damaging agent (e.g., radiation or a chemotherapeutic agent).

In one embodiment, the invention provides methods of killing tumor cell comprising contacting the cells with at least one small inhibitory RNA (siRNA) specific for a DNA repair protein and at least one DNA-damaging agent. In a preferred embodiment, the DNA repair protein is ATM, ATR, or DNA-PK_{cs}. In another preferred embodiment, the siRNA is encoded by a nucleic acid sequence selected from the group consisting of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

In one embodiment, the DNA-damaging agent is radiation. In another embodiment, the DNA-damaging agent is a chemotherapeutic agent, for example, an alkylating agent such as a nitrogen mustard (e.g., chlorambucil, cyclophosphamide, estramustine, ifosfamide, mechlorethamine, or melphalan), an aziridine (e.g., thiotepa), an alkyl sulfonate (e.g., busulfan), a nitrosurea (e.g., carmustine, lomustine, or streptozocin), a platinum complex (e.g., carboplatin or cisplatin), and a nonclassic alkylator (e.g., altretamine, dacarbazine, procarbazine, or temozolamide).

In another embodiment, the methods of the invention further comprise contacting the cell with at least a second chemotherapeutic agent.

In one embodiment, the tumor cells are derived from a cell or tissue type selected from the group consisting of prostate, colon, breast, lung, brain, skin, ovary, pancreas, liver, stomach, bladder, bone, testicle, uterus, adipose tissue, throat, kidney, tongue, pituitary gland, thyroid, nerve, lymphoid tissue, eye, or cervix. In a preferred embodiment, the tumor cells are resistant to killing by contacting with the DNA damaging agent alone.

In a preferred embodiment, the siRNA is expressed from a vector (e.g., a plasmid or an adenoviral vector).

In a further preferred embodiment, expression of the siRNA is controlled by a modified adenoviral promoter. Preferably, the modified adenoviral promoter comprises, in sequence, an adenoviral VA1 A-Box, at least one restriction enzyme recognition site, at least one adenoviral VA1 termination sequence, and at least one adenoviral VA1 B-box.

In another embodiment, the invention provides methods of treating a subject (e.g., a human) having cancer comprising administering to the subject a therapeutically effective amount of at least one small inhibitory RNA (siRNA) specific for a DNA repair protein and a therapeutically effective amount of at least one DNA-damaging agent.

In one embodiment, the DNA repair protein is AIM, ATR, or DNA-PK_{cs}. In another embodiment, the siRNA comprises a nucleic acid sequence selected from the group consisting of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

In one embodiment, the DNA-damaging agent is radiation. In another embodiment, the DNA-damaging agent is a chemotherapeutic agent, for example, an alkylating agent such as a nitrogen mustard (e.g., chlorambucil, cyclophosphamide, estramustine, ifosfamide, mechlorethamine, or melphalan), an aziridine (e.g., thiotepa), an alkyl sulfonate (e.g., busulfan), a nitrosurea (e.g., carmustine, lomustine, or streptozocin), a platinum complex (e.g., carboplatin or cisplatin), and a nonclassic alkylator (e.g., altretamine, dacarbazine, procarbazine, or temozolamide).

In another embodiment, the methods of the invention further comprise administering at least a second chemotherapeutic agent to the subject.

In one embodiment, the cancer is derived from a cell or tissue type selected from the group consisting of prostate, colon, breast, lung, brain, skin, ovary, pancreas, liver, stomach, bladder, bone, testicle, uterus, adipose tissue, throat, kidney, tongue, pituitary gland, thyroid, nerve, lymphoid tissue, eye, and cervix. In a preferred embodiment, the cancer is resistant to treatment by administration of the DNA damaging agent alone.

In a preferred embodiment, the siRNA is expressed from a vector (e.g., a plasmid or an adenoviral vector).

In a further preferred embodiment, expression of the siRNA is controlled by a modified adenoviral promoter. Preferably, the modified adenoviral promoter comprises, in sequence, an adenoviral VA1 A-Box, at least one restriction enzyme recognition site, at least one adenoviral VA1 termination sequence, and at least one adenoviral VA1 B-box. In one embodiment, the siRNA is administered to the subject systemically. In another embodiment, the siRNA is administered to the subject locally at the site of a tumor.

In another embodiment, the siRNA is administered prior to administration of the DNA-damaging agent. In another embodiment, the siRNA is administered at the same time as the DNA-damaging agent. In yet another embodiment, the DNA-damaging agent is administered prior to the siRNA.

In still another embodiment, the invention provides an isolated nucleic acid molecule comprising a modified adenoviral VA1 promoter, wherein the modified adenoviral promoter comprises, in sequence, an adenoviral VA1 A-Box, at least one restriction enzyme recognition site, at least one adenoviral VA1 termination sequence, and at least one adenoviral VA1 B-box.

In another embodiment, the invention provides nucleic acid molecules comprising the nucleic acid sequence of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

Other features and advantages of the invention will be apparent to those skilled in the art from the following detailed description and claims.

### BRIEF DESCRIPTION OF THE DRAWINGS

FIGS. 1A-F depicts siRNA-mediated down-regulation of ATM, ATR, and DNA-PK_{cs} proteins in DU 145 cells after 48-hour transfection with siRNA-encoding plasmids. FIGS. 1A, 1C, and 1E, Western blots for ATM, ATR, and DNA-P_{cs}, respectively. Membranes were probed with antibodies for target protein, and expression levels were normalized for loading by probing for β-actin. Protein expression for ATM, ATR, and DNA-PK_{cs}, was quantified using a BioRad Versa-Doc imager and Quantity One analysis software and expressed as a percentage compared with that calculated in untransfected cells (FIGS. 1B, 1D, and 1F, respectively). Given that the respective transfection efficiencies were calculated as −45, 40, and 50, ≦90% inhibition of target protein are evident in the transfected population.

FIGS. 2A-2D depict clonogenic survival of DU 145 and PC-3 cells after transient transfection with ATM and DNA-PK_{cs}-targeted, siRNA-encoding plasmids followed by treatment with ionizing radiation. DU 145 (FIGS. 2A and C) or PC-3 cells (Figures B and D) were irradiated 48-hours post-transfection. Corresponding transfection efficiencies were calculated for each experiment using FACS analysis of replica wells transfected with an EGFP-encoding plasmid. Transfection efficiencies for each were calculated as ˜45% (FIGS. 2A and 2B), ˜25% (FIG. 2C), and ˜30% (FIG. 2D).

FIG. 3 depicts clonogenic survival of DU 145 cells after transient transfection with ATR-targeted, siRNA-encoding plasmids and treatment with MMS. A corresponding transfection efficiency of ˜50% was calculated using FACS analysis of replica wells transfected with an EGFP-encoding plasmid.

FIGS. 4A-4F depict enhanced attenuation of target protein expression and radiation-mediated cell killing of DU 145 cells after FACS enrichment of cells transfected with ATM and DNA-PY_{cs}-targeted, siRNA-encoding plasmids. Cells were transfected with either an EGFP-expressing plasmid alone (untransfected) or together with pSHAG-1 and pATM-2 (FIGS. 4A-4B) or pDNA-PK-1 (FIGS. 4D-4E) and FACS-sorted 48-hour post-transfection to enrich the transfected population. FACS-sorted cells were immediately seeded for clonogenic survival assays (FIGS. 4B and 4E), and the remaining cells were used to obtain protein extracts for Western analyses (FIGS. 4A and 4D). Target protein expression is plotted as the normalized expression in EGFP+ve-sorted cells compared with EGFP-ve-sorted cells. FIGS. 4C and 4F, clonogenic survival curves for DU 145 cells respectively treated with DMSO or high, nontoxic concentrations of the PI3k inhibitor Wortmannin or specific competitive DNA-PK_{cs}, inhibitor LY294002 one hour before and 24 hours after irradiation.

FIG. 5 depicts a schematic representation of the Ad5 siRNA (ERASE) vectors for use in the production of siRNA-encoding adenovirus.

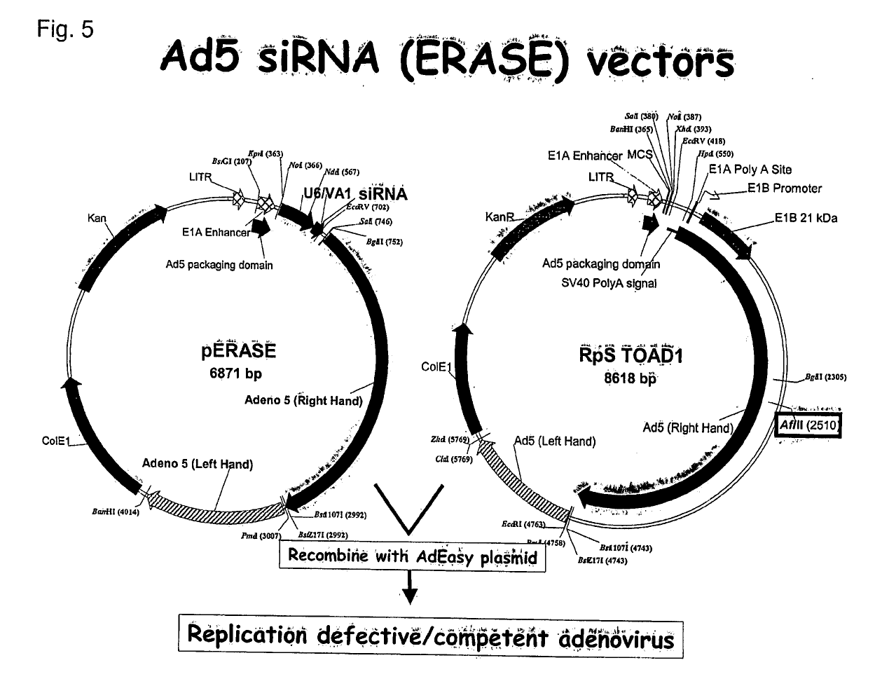

FIG. 6 depicts a schematic representation of an alternative pol III promoter (synthetic VA1). The natural adenovirus VA1 promoter is shown at the top, along with a schematic representation of the promoter bound to the transcription initiation complex (upper right). The modified promoter is shown at the bottom. Cloning sites for insertion of siRNA-encoding nucleic acid molecules, as well as the (T)₅ termination sequence, were inserted in between the A-Box and the B-Box of the natural promoter.

FIG. 7 depicts the effectiveness of the modified VA1 promoter, as compared to the U6 promoter, in PC-3 Luc cells.

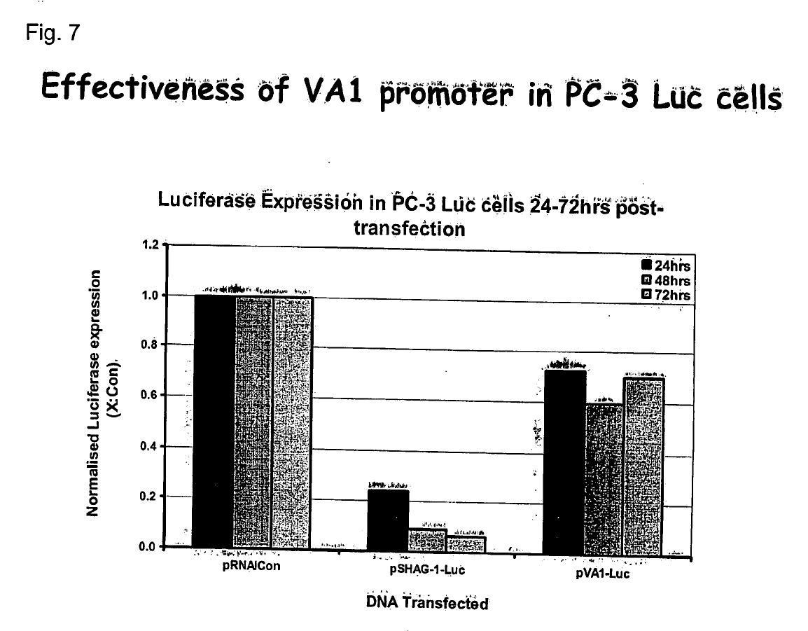

FIG. 8 depicts the effectiveness of the VA1 promoter, as compared to the U6 promoter, in 293 Luc cells.

FIG. 9 depicts the effectiveness of the VA1 promoter in DU 145 cells in cotransfection experiments.

FIG. 10 depicts the results of VA1 versus U6-mediated downregulation of DNA-PK in DU 145 cells.

FIG. 11 depicts the results of VA1 versus U6-mediated downregulation of DNA-PK in DU 145 cells at 72 hours post-transfection.

FIG. 12 depicts the results of VA1-mediated downregulation of DNA-PK in DU145 cells at 72 hours post-transfection.

FIG. 13 depicts the results of VA1 versus U6-mediated downregulation of DNA-PK in DU 145 cells at 72 and 96 hours post-transfection.

FIG. 14 depicts the sequences of the oligonucleotides used to construct the ATM-1 siRNA-encodingnucleic acid molecules. Oligo-A: SEQ ID NO:1. Oligo-B: SEQ ID NO:2. Target sequence: SEQ ID NO:3.

FIG. 15 depicts the sequences of the oligonucleotides used to construct the ATM-2 siRNA-encoding nucleic acid molecules. Oligo-A: SEQ ID NO:4. Oligo-B: SEQ ID NO:5. Target sequence: SEQ ID NO:6.

FIG. 16 depicts the sequences of the oligonucleotides used to construct the ATM-3 siRNA-encoding nucleic acid molecules. Oligo-A: SEQ ID NO:7. Oligo-B: SEQ ID NO:8. Target sequence: SEQ ID NO:9.

FIG. 17 depicts the sequences of the oligonucleotides used to construct the ATR-1 siRNA-encoding nucleic acid molecules. Oligo-A: SEQ ID NO:10. Oligo-B: SEQ ID NO:11. Target sequence: SEQ ID NO:12.

FIG. 18 depicts the sequences of the oligonucleotides used to construct the ATR-2 siRNA-encoding nucleic acid molecules. Oligo-A: SEQ ID NO:13. Oligo-B: SEQ ID NO:14. Target sequence: SEQ ID NO:15.

FIG. 19 depicts the sequences of the oligonucleotides used to construct the ATR-3 siRNA-encoding nucleic acid molecules. Oligo-A: SEQ ID NO:16. Oligo-B: SEQ ID NO:17. Target sequence: SEQ ID NO:18.

FIG. 20 depicts the sequences of the oligonucleotides used to construct the DNA-PK-1 siRNA-encoding nucleic acid molecules. Oligo-A: SEQ ID NO:19. Oligo-B: SEQ ID NO:20. Target sequence: SEQ ID NO:21.

FIG. 21 depicts the sequences of the oligonucleotides used to construct the DNA-PK-2 siRNA-encoding nucleic acid molecules. Oligo-A: SEQ ID NO:22. Oligo-B: SEQ ID NO:23. Target sequence: SEQ ID NO:24.

FIG. 22 depicts the sequences of the oligonucleotides used to construct the DNA-PK-3 siRNA-encoding nucleic acid molecules. Oligo-A: SEQ ID NO:25. Oligo-B: SEQ ID NO:26. Target sequence: SEQ ID NO:27.

FIG. 23 depicts siRNA-encoding nucleic acid sequences which can be used to target ATM, ATR, and DNA-PK in the methods of the invention. In order, the sequences are set forth as SEQ ID NOs:28-36.

FIG. 24 depicts the nuceleic acid sequence of the natural VA1 adenoviral promoter (SEQ ID NO:37). The locations of the variable upstream region, the +1 start site, the A-Box, and the B-box are indicated.

FIGS. 25A-25B depict two exemplary, non-limiting modified adenoviral VA1 promoters containing luciferase siRNA-encoding constructs. FIG. 25A: top strand: SEQ ID NO:38; bottom strand: SEQ ID NO:39. FIG. 25B: top strand: SEQ ID NO:40; bottom strand: SEQ ID NO:41.

### DETAILED DESCRIPTION OF THE INVENTION

The present invention is based, at least in part, on the discovery that inhibition of DNA repair protein expression through the use of small inhibitory RNA (siRNA) can augment radiation and chemotherapy-mediated killing of cancer cells. Accordingly, the present invention provides methods for killing cancer cells and methods of treating subjects having cancer, comprising administering siRNAs directed to DNA repair proteins (e.g., ATM, ATR and/or DNA-P_{cs}) in combination with one or more DNA-damaging agent (e.g., radiation or a chemotherapeutic agent).

RNA interference (RNAi) was first noted in Caenorhabditis elegans and plants as a novel mechanism of post-transcriptional gene silencing and has since been discovered in many eukaryotes (for reviews, see Hammond, S. M. et al. (2001) Nat. Rev. Genet. 2:110-119; and Zamore, P. D. (2001) Nat. Struct. Biol. 8:746-750). Rapid progress has been made in the use of RNAi and more specifically siRNAs as a means of attenuating the expression of specific proteins both in vitro and in vivo (Elbashir, S. M. et al. (2001) Nature, 411:494-498; Paddison, P. J. et al. (2002) Genes Dev. 16:948-958; Lewis, D. L. et al. (2002) Nat. Genet. 32:107-108; McCaffrey, A. P. et al. (2002) Nature, 418: 38-39) enabling any protein target, where the cDNA sequence is known, to be inhibited by these sequence-specific, double-stranded RNA molecules. For additional description of RNAi, see, for example, Scherr, M. et al. (2003) Curr Med Chem. 10(3):245-56; Shuey, D. J. et al. (2002) Drug Discov. Today 7(20):1040-6; Shi Y. (2003) Trends Genet. 19(1):9-12; Morita, T. and Yoshida, K. (2002) Tanpakushitsu Kakusan Koso 47(14):183945; Famulok, M. and Verma, S. (2002) Trends Biotechnol. 20(11):462-6; Timmons L. (2002) Mol Cell. 10(3):435-7; Kitabwalla, M, and Ruprecht, R. M. (2002) N. Engl. J. Med. 347(17):1364-7; McManus, M. T. and Sharp, P. A. (2002) Nat. Rev. Genet. 3(10):737-47; Micura, R. (2002) Angew Chem. Int. Ed. Engl. 41(13):2265-9; Lin, S. L. and Ying, S. Y. (2001) Curr. Cancer Drug Targets 1(3):241-7; Voinnet O. (2002) Curr. Opin. Plant Biol. 5(5):444-51; Cullen, B. R. (2002) Nat. Immunol. 3(7):597-9; Hudson, D. F. et al. (2002) Trends Cell Biol. 2(6):281-7; Mlotshwa, S. et al. (2002) Plant Cell 14 Suppl:S289-301; Ahlquist, P. (2002) Science 296(5571):1270-3; Ullu, E. et al. (2002) Philos. Trans. R. Soc. Lond. B. Biol. Sci. 357(1417):65-70; Inoue, H. (2001) Seikagaku 73(12):1444-7; Tuschl, T. (2001) Chembiochem. 2(4):239-45; and U.S. Patent Application Publication Nos. 20020086356, 20020132788, 20020173478.

Highly specialized DNA repair proteins together with upstream sensors and signalers protect mammalian cells from potentially lethal and/or tumorigenic lesions resulting from DNA damage (for recent reviews, see Jackson, S. P. (2001) Biochem. Soc. Trans. 29:655-661; Hoeijmakers, J. H. (2001) Nature, 411:366-374). Pivotal to these repair pathways are the DNA damage sensors ATM and ATR and DNA-PY_{cs}. The essential role these proteins play in DNA damage and repair is highlighted by the extreme sensitivity to DNA-damaging agents exhibited by cells and animal models defective in and/or lacking ATM, ATR, and DNA-PKcs expression (Miller, S. P. et al. (1995) Science, 267:1183-1185; Meyn, M. S. (1995) Cancer Res., 55: 5991-6001; Shiloh, Y. (2001) Curr. Opin. Genet. Dev. 11:71-77). Thus, targeted inhibition of these kinases is an attractive approach in the development of potent radiation therapy strategies. To increase the radio- and chemotherapy-mediated cell killing of human tumor cells, the present invention uses vector systems, including a plasmid-based pol III promoter system (Paddison, P. J. et al. (2002) Genes Dev. 16:948-958) and an adenoviral vector system, to deliver and express siRNAs targeted toward DNA repair proteins such as ATM, ATR, and DNA-PK_{cs}.

The data presented herein demonstrate the effective use of siRNA as a novel tool for modulating killing of human cancer cells by DNA-damaging agents, including radiation and chemotherapeutic agents. The inherent specificity of this approach provides a powerful method of target protein downregulation that can be incorporated into several existing viral and nonviral vector delivery platforms, including adenoviral vectors.

I. Definitions

As used herein, the terms “neoplastic cells”, “neoplasia”, “tumor”, “tumor cells”, “cancer” and “cancer cells” (used interchangeably) refer to cells which exhibit relatively autonomous growth, so that they exhibit an aberrant growth phenotype characterized by a significant loss of control of cell proliferation. Neoplastic cells can be malignant or benign.

As used herein, “suppressing tumor growth” refers to reducing the rate of growth of a tumor, halting tumor growth completely, causing a regression in the size of an existing tumor, eradicating an existing tumor and/or preventing the occurrence of additional tumors upon treatment with the compositions, kits or methods of the present invention. “Suppressing” tumor growth indicates a growth state that is curtailed when compared to growth by cells treated only with a DNA-damaging agent (e.g., radiation or chemotherapy), without treatment with the siRNA of the invention. Tumor cell growth can be assessed by any means known in the art, including, but not limited to, directly measuring tumor size, radiographic imaging, utilizing serum biomarkers of disease burden (e.g., serum PSA), determining whether tumor cells are proliferating using a ³H-thymidine incorporation assay or clonogenic assay, or counting tumor cells.

“Suppressing” tumor cell growth means any or all of the following states: slowing, delaying, and stopping tumor growth, as well as tumor shrinkage.

“Delaying development” of a tumor means to defer, hinder, slow, retard, stabilize, and/or postpone development of the disease. This delay can be of varying lengths of time, depending on the history of the disease and/or individual being treated.

As used herein, “synergy” or “synergistic effect” when referring to combination administration of siRNA and DNA-damaging agent and/or radiation means that the effect of the combination is more than additive when compared to administration of adenovirus vector, DNA-damaging agent or radiation alone.

An “adenovirus vector” or “adenoviral vector” (used interchangeably) used in the methods of the invention may be in any of several forms, including, but not limited to, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. For purposes of this invention, adenovirus vectors are replication-competent in a target cell.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

The terms “polynucleotide” and “nucleic acid”, used interchangeably herein, refer to a polymeric form of nucleotides of any length, either ribonucleotides and/or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18. A preferred alignment program is ALIGN Plus (Scientific and Educational Software, Pennsylvania), preferably using default parameters, which are as follows: mismatch=2; open gap=0; extend gap=2.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

The term “heterologous” means a DNA sequence not found in the native vector genome. With respect to a “heterologous transcriptional regulatory sequence”, “heterologous” indicates that the transcriptional regulatory sequence is not naturally ligated to the DNA sequence for the gene essential for replication of the vector.

The term “promoter” is used according to its art-recognized meaning. It is intended to mean the DNA region, usually upstream to the coding sequence of a gene or operon, which binds RNA polymerase and directs the enzyme to the correct transcriptional start site.

The term “replication” means duplication of a vector. This duplication, in the case of viruses, can occur at the level of nucleic acid, or at the level of infectious viral particle. In the case of DNA viruses, replication at the nucleic acid level comprises DNA replication. In the case of RNA viruses, nucleic acid replication comprises replication into plus or minus strand (or both). In the case if retroviruses, replication at the nucleic acid level includes the production of cDNA as well as the further production of RNA viral genomes. The essential feature is the generation of nucleic acid copies of the original viral vector. However, replication also includes the formation of infectious DNA or RNA viral particles. Such particles may successively infect cells in a given target tissue, thus distributing the vector through all or a significant portion of the target tissue.

In the context of adenovirus, a “heterologous polynucleotide” or “heterologous gene” or “transgene” is any polynucleotide or gene that is not present in wild-type adenovirus. Preferably, the transgene will also not be expressed or present in the target cell prior to: introduction by the adenovirus vector.

In the context of adenovirus, a “heterologous” promoter or enhancer is one which is not associated with or derived from an adenovirus gene.

A “host cell” includes an individual cell or cell culture which can be or has been a recipient of an adenoviral vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with an adenoviral vector of this invention.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “individual” is a vertebrate, preferably a mammal, more preferably a human. Mammals include, but are not limited to, farm animals, sport animals, rodents, primates, and pets

An “therapeutically effective amount” is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

“Expression” includes transcription and/or translation.

II. Methods of the Invention

In one embodiment, the invention provides methods of treating a subject (e.g., a human) having cancer comprising administering to the subject a therapeutically effective amount of at least one small inhibitory RNA (siRNA) specific for a DNA repair protein and a therapeutically effective amount of at least one DNA-damaging agent.

As used herein, “treatment” of a subject includes the application or administration of a therapeutic agent (e.g., an siRNA and/or a DNA-damaging agent) to a subject, or application or administration of a therapeutic agent to a cell or tissue from a subject, who has a diseases or disorder (e.g., cancer), has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

In some embodiments, the methods of the present invention provide for suppressing tumor growth. In other embodiments, the methods are for reducing size and/or extent of a tumor. In other embodiments, the methods are for delaying development of a tumor. In other embodiments, the methods are for treating a neoplasia. In still other embodiments, the methods are for killing tumor cells.

With respect to all methods described herein, target cancer cells (i.e., neoplastic, proliferative cells) are contacted with an appropriate siRNA vector described herein (preferably in the form of an adenovirus) such that the vector enters the cell and expression of the siRNA is induced. The target cancer cells are further exposed to a DNA-damaging agent (e.g., radiation and/or chemotherapeutic agent(s)).

Individuals suitable for treatment by these methods include individuals who have or are suspected of having cancer, including individuals in the early or late stages of the disease, as well as individuals who have previously been treated or are about to undergo treatment (e.g., are in the adjuvant or neoadjuvant setting). Other individuals suitable for the methods described herein are those who are considered high risk for developing a tumor, such as those who have a genetic predisposition to development of a neoplasia and/or who have been exposed to an agent(s) which is correlated with development of a neoplasia. In one embodiment, the methods of the present invention are particularly useful for the treatment of tumors that are resistant to at least one form of cancer therapy, including radiation and/or chemotherapy.

The presence of cancer and the suitability of the individual for receiving the methods described herein may be determined by any of the techniques known in the art, including diagnostic methods such as imaging techniques, analysis of serum tumor markers, and biopsy.

The siRNA may be targeted to any DNA repair protein known to participate in DNA repair pathways activated in response to DNA-damaging agents, including, but not limited to, ATM, ATR, and/or DNA-PK_{cs}. The siRNA may target any region in the target mRNA, and may be encoded, for example, by one ore more of the nucleic acid sequences set forth in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36. The methods of the invention may use siRNA targeting a single DNA repair protein, or may use a mixture of siRNA targeting more than one DNA repair protein. For example, in a preferred embodiment, the methods of the invention use siRNA targeting one, two, three or more DNA repair proteins.

As used herein, a “DNA-damaging agent” is any agent or treatment that, when administered to a cell or a subject, e.g., a human subject, cause damage to the cell or subject's DNA (e.g., genomic DNA). In one embodiment, the DNA-damaging agent is radiation. In another embodiment, the DNA-damaging agent is a chemotherapeutic agent. Preferred chemotherapeutic DNA-damaging agents include, but are not limited to, alkylating agents such as nitrogen mustards (e.g., chlorambucil, cyclophosphamide, estramustine, ifosfamide, mechlorethamine, and melphalan), aziridines (e.g., thiotepa), alkyl sulfonates (e.g., busulfan and methyl methanesulfonate (MMS)), nitrosureas (e.g., carmustine, lomustine, and streptozocin), platinum complexes (e.g., carboplatin and cisplatin), and nonclassic alkylators (e.g., altretamine, dacarbazine, procarbazine, and temozolamide). In some embodiments, the methods of the present invention comprise the use of one or more DNA-damaging agents.

In another embodiment, the methods of the invention can be administered in conjunction with other known treatments for cancer, including, but not limited to, mechanical removal of cancerous cells (e.g., surgical removal of a tumor), and administration of chemotherapeutic agents. In addition to DNA-damaging agents, there are many other chemotherapeutic agents used to treat cancer which act to kill cancer cells and/or slow their growth through other mechanisms. The administration of such additional treatments and/or agents are intended to be included in the methods of the present invention.

For example, other chemotherapeutic agents that may be used in conjunction with the methods of the invention include, but are not limited to, antimetabolites such as folate analogs (e.g., methotrexate), purine analogs (e.g., fludarabine, mercaptopurine, and thioguanine (e.g., 6-TG)), adenosine analogs (e.g., cladribine, and pentostatin), pyrimidine analogs (e.g., capecitabine, cytarabine, depocyt, floxuridine, fluorouracil (e.g., 5-FU); and gemcitabine), and substituted ureas (e.g., hydroxyurea); natural products such as antitumor antibiotics (e.g., bleomycin, dactinomycin, actinomycin D, daunorubicin, daunomycin, DaunoXome (liposomal daunorubicin), doxorubicin, Doxil (liposomal-doxorubicin), epirubicin, idarubicin, mitoxantrone, and mitomycin C), epipodophyllotoxins (e.g., etoposide and teniposide), microtuble agents (e.g., docetaxel, paclitaxel, vinblastine, vincristine, and vinorelbine), camptothecin analogs (e.g., irinotecan and topotecan), enzymes (e.g., asparaginase), and monoclonal antibodies (e.g., alemtuzamab, gemtuzumab ozogamicin, ibritumomab tiuxetan, nofetumomab, rituximab, tositumomab, and trastuzumab). Any of these agents which have DNA-damaging activity may also be used directly in the methods of the invention.

Still further chemotherapeutic agents that may be used in conjunction with the methods of the invention include, but are not limited to, aldesleukin, alitretinoin, allopurinol, altretamine, amifostine, anastrozole, arsenic trioxide, bexarotene, calusterone, capecitabine, celecoxib, cimetidine, darbepoetin alfa, denileukin diflitox, dexamethazone, dexrazoxane, diphenhydramine, dromostanolone propionate, epoetin alfa, estramustine, exemestane, filgrastim, floxuridine, fludarabine, flutamide, fulvestrant, goserelin, imatinib mesylate, interferon alfa-2a, interferon alfa-2b, letrozole, leucovorin, leuprolide, levamisole, megestrol acetate, mercaptopurine (e.g., 6-MP), mesna, methoxsalen, mitotane, nandrolone phenpropionate, oprelvekin, oxaliplatin, pamidronate, pegademase, pegaspargase, pegfilgrastim, pentostatin, pipobroman, plicamycin, mithramycin, porfimer sodium, prednisone, quinacrine, ranitidine, rasburicase, sargramostim, talc, tamoxifen, testolactone, toremifene, tretinoin, uracil mustard, valrubicin, and zoledronate. Those of skill in the art will recognize that any of these chemotherapeutic agents may fit into one or more particular classes of chemotherapeutic agents described above, including DNA-damaging agents.

Those of skill in the art will appreciate that preferred dosage levels and schedules are well-known in the field of cancer treatment. Accordingly, in one embodiment, the methods of the invention will be used in accordance with standard dosage levels and schedules for the DNA-damaging agents. A useful general resource for methods of treating and managing cancer is Pazdur, R. et al., eds., Cancer Management: A Multidiscliplinary Approach, 7^{th} edition. The Oncology Group, a division of SCP Communications, Inc., New York:2003. Non-limiting exemplary chemotherapy and radiation treatment regimens may be further found, for example, in Bonadonna, G. et al. (1976) N. Engl. J. Med. 294:405-410; Bonadonna, G. et al., in Salmon SE (ed): Adjuvant Therapy of Cancer VI, pp 169-173, 1990; Weiss, R. B. et al. (1987) Am J Med 83:455-463; Budman, D. R. et al. (1992) Proc. Am. Soc. Clin. Oncol. 11:51; Hortobagyi, G. N. et al. (1979) Cancer 43:1225-1233; Fisher, B. et al. (1990) J. Clin. Oncol. 8:1483; Henderson, I. C. et al. (1998) Proc. Am. Soc. Clin. Oncol. 17:101A; Slamon, D. et al. (1998) Proc. Am. Soc. Clin. Oncol. 17:377A; Norton, L. et al. (1999) Proc. Am. Soc. Clin. Oncol. 18:483A; Pouillart, P. et al. (1999) Proc. Am. Soc. Clin. Oncol. 18:2275A; Nabholtz, J. M. et al. (1999) Proc. Am. Clin. Oncol. 18:485A; Seidman, A. D. et al. (2000) Proc. Am. Soc. Clin. Oncol. 19:319A; Seidman, A. D. et al. (1995) J. Clin. Oncol. 13:2575-2581; Ravdin, P. M. et al. (1995) J. Cin. Oncol. 13:2879-2885; Vogel, C. et al. (2000) Proc. Am. Soc. Clin. Oncol. 19:275A; Michaud, L. et al. (2000) Proc. Am. Soc. Clin. Oncol. 19:402A; Leichman, L. et al. (1985) Am. J. Med. 78:211-215; Wagner, J. P. et al. (1994) Int. J. Radiat. Oncol. Biol. Phys. 29:17-23; Poon, M. A. et al. (1989) J. Clin. Oncol. 7:1407-1418; Moertel, C. G. et al. (1995) Ann. Intern. Med. 122:321-326; Moertel, C. G. et al. (1990) N. Engl. J. Med. 322:352-358; Wolmark, N. et al. (1996) Proc. Am. Soc. Clin. Oncol. 15:205; Moertel, C. G. et al. (1994) J. Clin. Oncol. 12:21-27; Saltz, L. B. et al. (1999) Proc. Am. Soc. Clin. Oncol. 18:898A; Saltz, L. B. et al. (2000) N. Engl. J. Med. 343:905?14; Pitot, H. C. et al. (1997) J. Clin. Oncol. 15:2910-2919; Cunningham, D. et al. (1998) Proc. Am. Soc. Clin. Oncol. 17:1A; Twelves, C. et al. (1999) Proc. Am. Soc. Clin. Oncol. 18:1010A; Herskovic, A. et al. (1992) N. Engl. J. Med. 326:1593-1598; Preusser, P. et al. (1989) J. Clin. Oncol. 7:1310-1317; Lerner, A. et al. (1992) J. Clin. Oncol. 10:536-540; Kelsen, D. et al. (1992) J. Clin. Oncol. 10:541-548; Macdonald, J. S. et al. (2000) Proc. Am. Soc. Clin. Oncol. 19:1 A; Gastrointestinal Tumor Study Group (1987) Cancer 59:2006-2010; Moertel, C. G. et al. (1994) J. Clin. Oncol. 12:21-27; Burris, H. A. et al. (1997) J. Clin. Oncol. 15:2403-2413; Sternberg, J. J. et al. (1977) JAMA 238:2282-2287; Loehrer, P. J. et al. (1992) J. Clin. Oncol. 10:1066-1073; Redman, B. et al. (1997) Proc. Am. Soc Clin. Oncol. 16:325A; Kaufinan, D. et al. (1998) Proc Am. Soc. Clin. Oncol. 17:320A; Burch, P. A. et al. (1999) Proc. Am. Soc. Clin. Oncol. 18:1266A; Vaishampayan, U. et al. (1999) Proc. Am. Soc. Clin. Oncol. 18:1282A; Pienta, K. J. et al. (1994) J. Clin. Oncol. 12:2005-2012; Tannock, I. F. et al. (1996) J. Clin. Oncol. 14:1756-1764; Crawford, E. D. et al. (1987) N. Engl. J. Med. 32:419-424; Jurincic, C. D. et al. (1991) Semin. Oncol. 18(suppl 6):21-25; Fyfe, G. et al. (1995) J. Clin. Oncol. 13:688-696; Yang, J. C. et al. (1998) J. Clin. Oncol. 12:1572-1576; Williams, S. D. et al. (1987) N. Engl. J. Med. 316:1435-1440; Loehrer, P. J. et al. (1988) Ann. Intern. Med. 109:540-546; Miller, K. D. et al. (1997) J. Clin. Oncol. 15:1427-1431; Loehrer, P. J. et al. (1988) Ann. Intern. Med. 109:540-546; Murad, A. M. et al. (1992) Proc. Am Soc Clin Oncol 11:229; Morris, M. et al. (1999) N. Engl. J. Med 340:1137-1143; Rose, P. G. et al. (1999) N. Engl. J. Med. 340:1144-1153; Albert, D. et al. (1992) J. Clin. Oncol. 10:706-717; McGuire, W. P. et al. (1996) N Engl J Med 334:1-6; Coleman, R. L. et al. (1997) Cancer J. Sci. Am. 3:246-253; Iva, B. et al. (2000) Proc. Am. Soc. Clin. Oncol. 19:1570A; Muggia, F. M. et al. (1997) J. Clin. Oncol. 15:987-993; Jacobs, C. et al. (1992) J. Clin. Oncol. 10:257-263; Dang, T. P. et al. (1998) Proc. Am. Soc. Clin. Oncol. 17:393a; Roa, V. et al. (1996) Proc. Am. Soc. Clin. Oncol. 15:A1231; Wozniak, A. J. et al. (1998) J. Clin. Oncol. 16:2459-2465; Longeval, E. and Klastersky, J. (1982) Cancer 50:2751-2756; Sandler, A. et al. (1998) Proc. Am. Soc. Clin. Oncol. 17:454A; Ricci, S. et al. (1999) Proc. Am. Soc. Clin. Oncol. 18:480A; Le Chevalier, T. et al. (1994) J. Clin. Oncol. 12:360-367; Shephaerd, F. A. et al. (2000) J. Clin. Oncol. 18:2095-2103; Vansteenkiste, J. et al. (2000) Proc. Am. Soc. Clin. Oncol. 19:1910A; Shin, D. M. et al. (1998) Ann. Intern. Med. 129:100-104; Skarlos, D. V. et al. (1994) Ann. Oncol. 5:601-607; Rosen, G. et al. (1983) J. Cancer Res. Clin. Oncol. 106:55-67; Pratt, C. B. et al. (1985) Cancer 56:1930-1933; Rosen, G. et al. (1981) Cancer 47:2204-2213; Pinedo, H. M. et al. (1984) Cancer 53:182-183; Elias, A. et al. (1989) J. Clin. Oncol. 7:1208-1216; Creagan, E. T. et al. (1999) J. Clin. Oncol. 17:1884-1890; Capizzi, R. L. (1999) Semin. Oncol. 26(suppl 7):1-2; Perry, M. C. (1992) Semin. Oncol. 19(5):453-7, 551-65; Wadler, S et al. (1998) J. Clin. Oncol. 16(9):3169-78, 1998; all of which are incorporated herein by reference.

Because the administration of the siRNA sensitizes cancer cells to DNA-damaging agents, the methods of the invention may also preferably use lower and/or less-frequent doses of DNA-damaging agents than would be used if the DNA-damaging agents were being administered without the siRNA. Use of lower and/or less-frequent doses of DNA-damaging agents is highly desirable, because such agents frequently have undesirable side-effects. Additionally, the methods of the invention are particularly useful in the treatment of cancer that are resistant conventional chemotherapeutic agents (including DNA-damaging agents) and radiation, because treatment of cancer cells with the siRNAs of the invention renders the cells more sensitive to DNA-damaging agents.

The methods of the invention are intended to be used for any type of tumor, cancer, and/or neoplasm, including, but not limited to, those derived from prostate, colon, breast, lung, brain, skin, ovary, pancreas, liver, stomach, bladder, bone, testicle, uterus, adipose tissue, throat, kidney, tongue, pituitary gland, thyroid, nerve, lymphoid tissue, eye, and/or cervix. Additionally, the methods of the invention are intended to be used for tumors which may be a mixture of more than one cell type, as well as for metastasized tumors which are originally derived from one cell type, but have migrated to a different part of the body.

Although methods of tumor suppression are exemplified in the discussion below, it is understood that the alternative methods, described above are equally applicable and suitable, and that the endpoints of these methods (e.g., efficacy of treatment) are measured using methods standard in the art, including the diagnostic and assessment methods described above.

Delivery of adenoviral vectors is discussed infra and is generally accomplished by either site-specific injection (local administration) or intravenously (systemic administration). Direct intra-tumor injections are preferred. Site-specific injections of either vector may include, for example, injections into the portal vein of the liver as well as intraperitoneal, intrapleural, intrathecal, intra-arterial, intra-tumor injections or topical application. These methods are easily accommodated in treatments using adenoviral vectors.

The adenoviral vectors may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular adenoviral vector (including its form) as well as the type and location of the target cells (i.e., whether the cells are to be transfected or transformed in vitro or in vivo). If used as a packaged adenovirus, adenovirus vectors may be administered in an appropriate physiologically acceptable carrier at a dose of about 1 to about 10. The multiplicity of infection will generally be in the range of about 0.001 to 100. If administered as a polynucleotide construct (i.e., not packaged as a virus) about 0.01 μg to about 1000 μg of an adenoviral vector can be administered. The adenoviral vector(s) may be administered one or more times, depending upon the intended use and the immune response potential of the host, and may also be administered as multiple, simultaneous injections. If an immune response is undesirable, the immune response may be diminished by employing a variety of immunosuppressants, so as to permit repetitive administration, without a strong immune response. If packaged as another viral form, such as HSV, an amount to be administered is based on standard knowledge about that particular virus (which is readily obtainable from, for example, published literature) and can be determined empirically.

Administration of the above-described methods may also include repeat doses or courses of target-cell specific adenovirus depending, inter alia, upon the individual's response and the characteristics of the individual's disease. Repeat doses may be undertaken immediately following the first course of treatment (i.e., within one day), or after an interval of days, weeks or months to achieve and/or maintain suppression of tumor growth.

Generally, an effective amount of adenovirus vector is administered, i.e., amounts sufficient to achieve the desired result, based on general empirical knowledge of a population's response to such amounts. Some individuals are refractory to these treatments, and it is understood that the methods encompass administration to these individuals. The amount to be given depends, inter alia, on the stage of the cancer, the condition of the individual, the extent of disease, the route of administration, how many doses will be administered, and the desired objective.

III. Isolated Nucleic Acid Molecules

One aspect of the invention pertains to isolated nucleic acid molecules that encode the siRNAs used in the methods of the invention. The invention further pertains nucleic acid molecules comprising the modified adenoviral VA1 promoter. As used herein, the term ‘nucleic acid molecule’ is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

In general, optimal practice of the present invention can be achieved by use of recognized manipulations. For example, techniques for isolating mRNA, purifying and analyzing nucleic acids, methods for making recombinant vector DNA, cleaving DNA with restriction enzymes, ligating DNA, introducing DNA into host cells by stable or transient means, culturing the host cells, producing recombinant adenoviral vectors, and isolating and purifying polypeptides a are generally known in the field. See generally Sambrook et al., Molecular Cloning (2d ed. 1989), and Ausubel et al., Current Protocols in Molecular Biology, (1989) John Wiley & Sons, New York.

The term ‘isolated nucleic acid molecule’ includes nucleic acid molecules which are separated from other nucleic acid molecules which are present in the natural source of the nucleic acid. For example, with regards to genomic DNA, the term ‘isolated’ includes nucleic acid molecules which are separated from the viral DNA or chromosome with which the genomic DNA is naturally associated. Preferably, an ‘isolated’ nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated siRNA-encoding nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the DNA repair protein that the siRNA targets. Moreover, an ‘isolated’ nucleic acid molecule can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 2, 4, 5, 7, 8, 10, 11, 13, 14, 16, 17, 19, 20, 22, 23, 25, 26, 28, 29, 30, 31, 32, 33, 34, 35; or 36, or a portion thereof, can be constructed using standard molecular biology techniques and the sequence information provided herein.

Moreover, a nucleic acid molecule encompassing all or a portion of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, or 3 can be isolated by the polymerase chain reaction (PCR) using synthetic oligonucleotide primers designed based upon the sequence of NO: 1, 2, 4, 5, 7, 8, 10, 11, 13, 14, 16, 17, 19, 20, 22, 23, 25, 26, 28, 29, 30, 31, 32, 33, 34, 35, or 36.

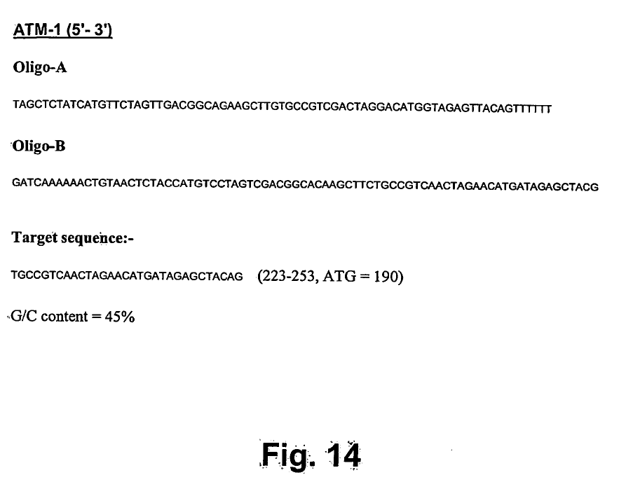

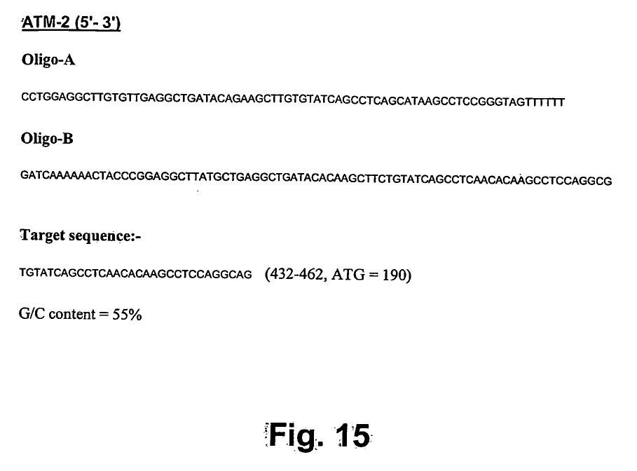

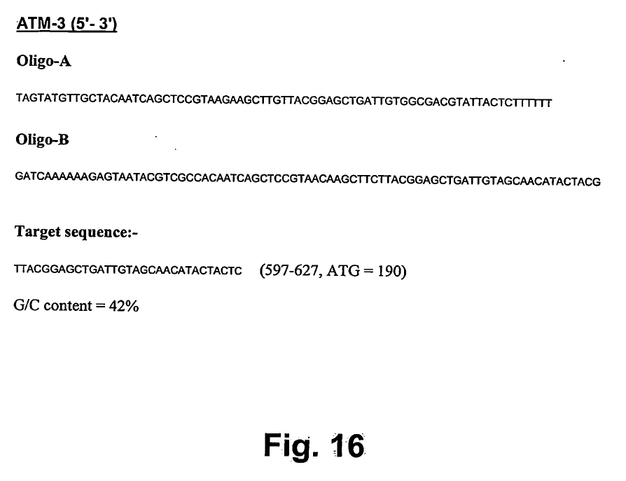

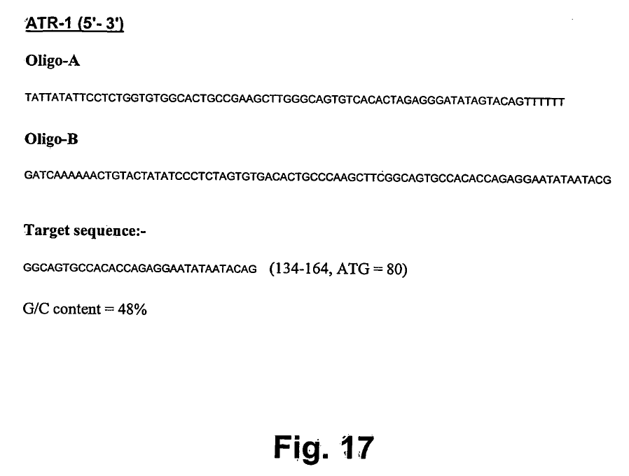

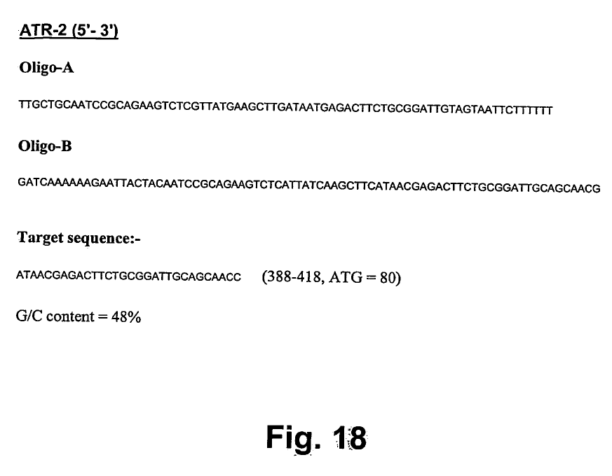

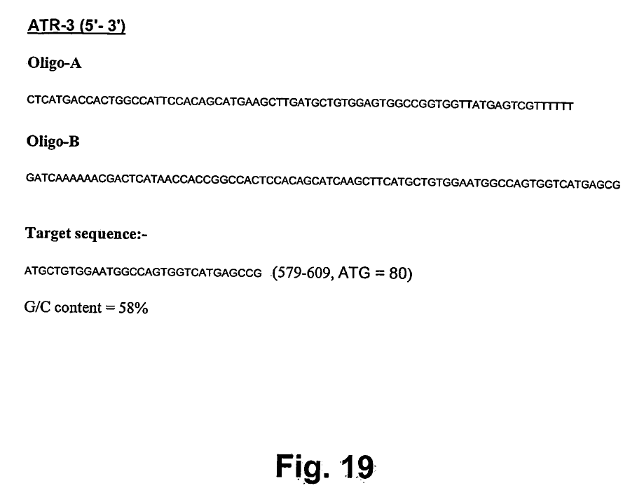

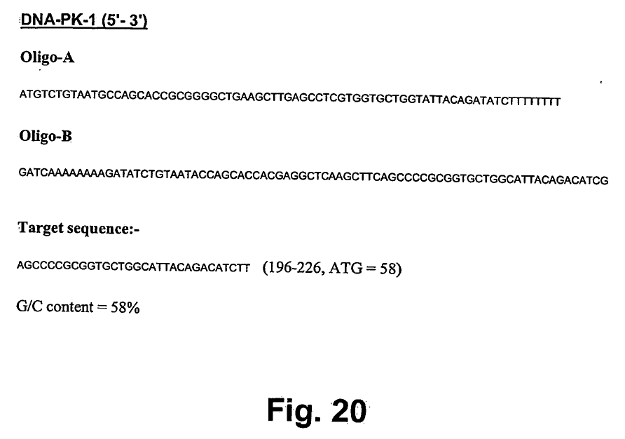

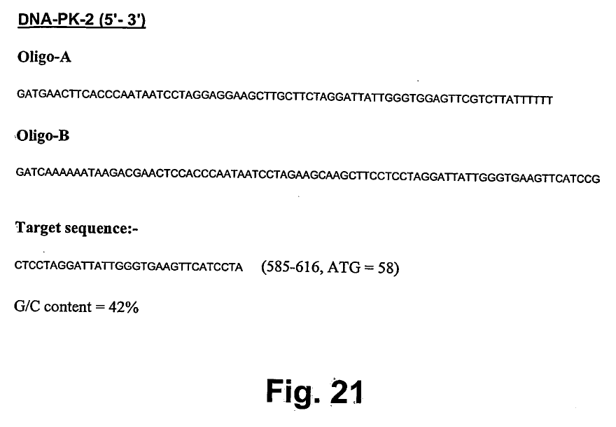

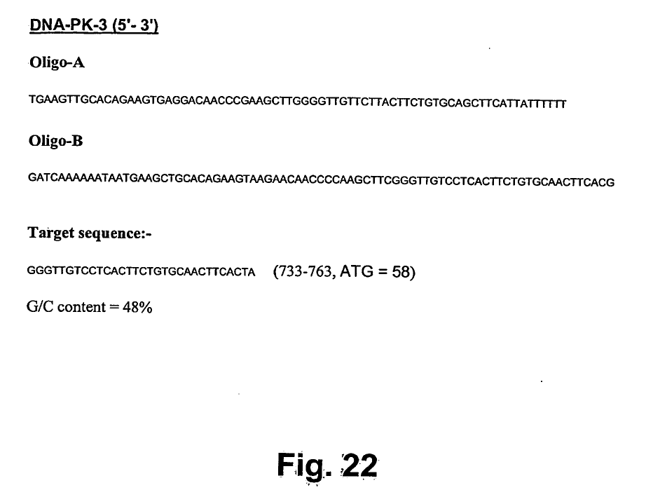

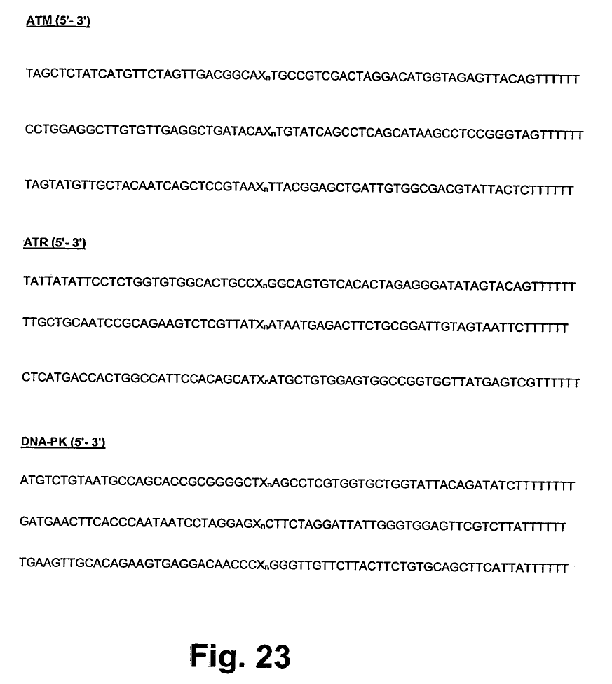

In a preferred embodiment, the siRNAs used in the methods of the invention are produced by inserting a double-stranded DNA molecule that encodes the siRNA into an expression vector (e.g., a plasmid or an adenoviral vector), such that the siRNA can be expressed in a cell (e.g., a cancer cell). In one embodiment, the siRNAs are encoded by the nucleic acid sequence of SEQ ID NO:1,2, 4, 5, 7, 8, 10, 11, 13, 14, 16, 17, 19, 20, 22, 23, 25, 26, 28, 29, 30, 31, 32, 33, 34, 35, or 36 (see FIGS. 14-23). In a preferred embodiment, the siRNAs used in the methods of the invention are encoded by SEQ ID NOs:28-36 (FIG. 23), wherein ‘X’ may be any nucleotide (e.g., A, T, C, or G), and ‘Xₙ’ represents a flexible number of nucleotides, wherein ‘n’ may be any number, provided that the nucleotides represented by ‘Xₙ’ are capable of forming a loop in the siRNA hairpin structure. In a preferred embodiment, n=8.

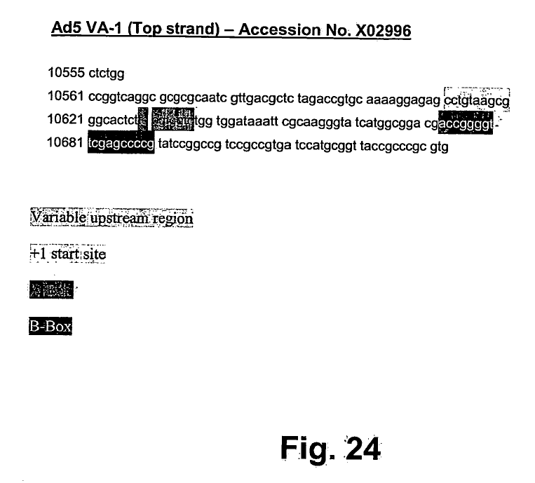

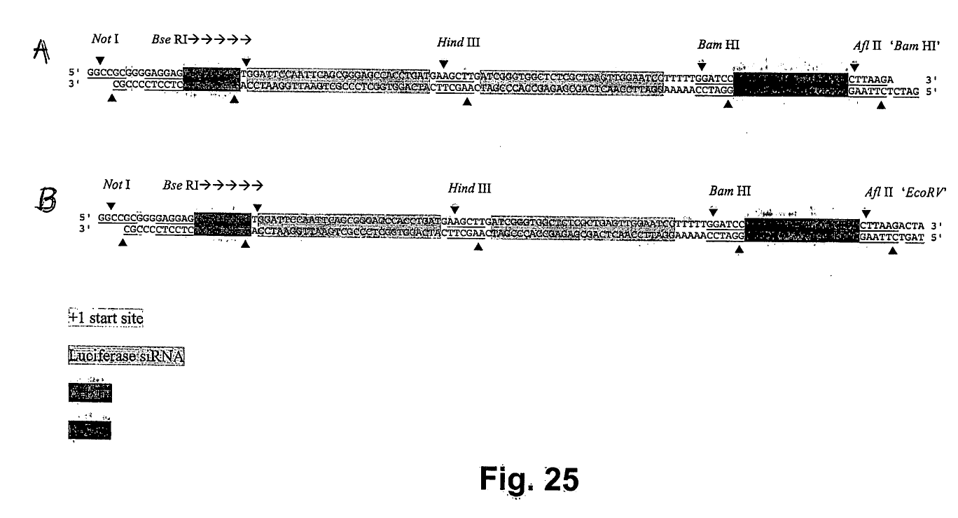

In another embodiment, an isolated nucleic acid molecule of the invention is a modified VA1 promoter, which comprises, in sequence, an adenoviral VA1 A-Box, at least one restriction enzyme recognition site, at least one adenoviral VA1 termination sequence, and at least one adenoviral VA1 B-box. A nucleic acid molecule encoding an siRNA can be subcloned in between the A and B boxes. The sequence of the wild-type (natural) VA1 promoter is shown in FIG. 24. Exemplary, non-limiting modified adenoviral VA1 promoters containing luciferase siRNA-encoding constructs are shown in FIGS. 25A-25B. Further description of the adenoviral VA1 promoter can be found, for example, in Lai, C. M. et al. (2002) Exp Eye Res. 75(6):625-34; Cagnon, L et al. (1995) J. Acquir. Immune Defic. Syndr. Hum. Retrovirol. 9(4):349-58; Jennings, P. A. and Molloy, P. L. (1987) EMBO J. 6(10):3043-7; Paule, M. R and White, R. J. (2000) Nucleic Acids Res. 28(6): 1283-98; Fowlkes, D. M. and Shenk, T. (1980) Cell 22(2 Pt 2):405-13; Yu, M et al. (1995) Virology 206(1):381-6; and Ma, Y. and Mathews, M. B. (1996) J. Virol. 70(8):5083-99.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to siRNA nucleotide sequences can be prepared by standard synthetic techniques, e.g., using an automated DNA synthesizer.

In still another embodiment, an isolated nucleic acid molecule of the invention comprises a nucleic acid molecule which is a complement of the nucleotide sequence shown in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36. A nucleic acid molecule which is complementary to the nucleotide sequence shown in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36 is one which is sufficiently complementary to the nucleotide sequence shown in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36 such that it can hybridize to the nucleotide sequence shown in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, thereby forming a stable duplex. The term ‘complementary’ or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99 to about 100%. Complementary polynucleotide sequences can be identified by a variety of approaches including use of well-known computer algorithms and software.

In still another embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to the nucleotide sequence shown in SEQ ID NO:1, 2, 4, 5, 7, 8, 10, 11, 13, 14, 16, 17, 19, 20, 22, 23, 25, 26, 28, 29, 30, 31, 32, 33, 34, 35, or 36 (e.g., to the entire length of the nucleotide sequence), or a portion or complement of any of these nucleotide sequences. In one embodiment, a nucleic acid molecule of the present invention comprises a nucleotide sequence which comprises part or all of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, or a complement thereof, and which is at least (or no greater than) 25, 30, 50, 75, 100 or more nucleotides (e.g., contiguous nucleotides) in length.

To determine the percent identity of two nucleic acid or amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (for example, when aligning a second sequence to a nucleotide sequence having 100 nucleotides, at least 30, preferably at least 40, more preferably at least 50, even more preferably at least 60, and even more preferably at least 70, 80, or 90 nucleotides are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at online through the Genetics Computer Group), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at online through the Genetics Computer Group), using a NWSgapdnaCMP matrix and a gap weight of 40, 50, 60, 70, or 80 and a length weight of 1, 2, 3, 4, 5, or 6. A preferred, non-limiting example of parameters to be used in conjunction with the GAP program include a Blossum 62 scoring matrix with a gap penalty of 12, a gap extend penalty of 4, and a frameshift gap penalty of 5.

The nucleic acid molecule of the invention can comprise only a portion of the nucleic acid sequence of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, for example, a fragment which can be used as a probe or primer. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, or a complement thereof.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the siRNA or modified adenoviral VA1 promoter nucleotide sequences can be used to detect (e.g., specifically detect) siRNA or modified adenoviral VA1 promoter sequences. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a nucleotide sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which contain the expression construct, or which express the expressible sequence.

In another embodiment, nucleic acid molecules of the invention can comprise variants of the sequence elements disclosed herein. Nucleic acid variants (e.g., variants of the siRNA sequence used to target specific DNA repair protein mRNA) can be naturally occurring, such as allelic variants (same locus), homologues (different locus), and orthologues (different organism, e.g., mouse) or can be non-naturally occurring. Non-naturally occurring variants can be made by mutagenesis techniques, including those applied to polynucleotides, cells, or organisms. The variants can contain nucleotide substitutions, deletions, inversions and insertions. Allelic variants result, for example, from DNA sequence polymorphisms within a population (e.g., the human population).

As used herein, the term ‘hybridizes under stringent conditions’ is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na+] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

In addition to naturally-occurring variants of the sequences described herein that may exist in the population, the skilled artisan will further appreciate that changes can be introduced by mutation into the nucleotide sequences of SEQ ID NO:1, 2, 4, 5, 7, 8, 10, 11, 13, 14, 16, 17, 19, 20, 22, 23, 25, 26, 28, 29, 30, 31, 32, 33, 34, 35, or 363, without altering the functional ability of the siRNA sequences or the modified adenoviral VA1 promoter. In one embodiment, the isolated nucleic acid molecule comprises a nucleotide sequence which is at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:1, 2, 4, 5, 7, 8, 10, 11, 13, 14, 16, 17, 19, 20, 22, 23, 25, 26, 28, 29, 30, 31, 32, 33, 34, 35, or 36, e.g., to the entire length of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

IV. Recombinant Expression Vectors and Host Cells

Another aspect of the invention pertains to vectors, for example recombinant expression vectors, containing an siRNA-encoding nucleic acid molecule. As used herein, the term. ‘vector’ refers to a nucleic acid molecule capable of transporting another nucleic acid to which it has been linked. One type of vector is a ‘plasmid’, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as ‘expression vectors’. In general, expression vectors of utility in recombinant DNA techniques are often in the form of plasmids. In the present specification, ‘plasmid’ and ‘vector’ can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In a preferred embodiment, the siRNA-encoding nucleic acid molecules are contained within an adenoviral vector, which can be used to infect mammalian cells (e.g., human cells). In a more preferred embodiment, the adenoviral vector is replication incompetent. For techniques related to adenovirus, see, inter alia, Felgner and Ringold (1989) Nature 337:387-388; Berkner and Sharp (1983) Nucleic Acids Res. 11:6003-6020; Graham (1984) EMBO J. 3:2917-2922; Bett et al. (1993) J. Virology 67:5911-5921; Bett et al. (1994) Proc. Natl. Acad. Sci. USA 91:8802-8806. A preferred adenoviral vector system is the AdEasy system (He, T.-C. et al. (1988) Proc. Natl. Acad. Sci. USA 95:2509-2514; Zeng, M. et al. (2001) Biotechniques 31:260-262).

Adenoviruses are non-enveloped, regular icosohedral, double-stranded DNA viruses. For a complete review on adenoviruses and their replication, see Horwitz, M. S., Virology 2d ed, Fields, B. N., eds., Raven Press Limited, New York (1990), Chapter 60, pp. 1679-1721. Publications describing various aspects of adenovirus biology and/or techniques relating to adenovirus include the following: PCT/US95/14461; Graham and Van de Eb (1973) Virology 52:456467; Takiffet al. (1981) Lancet 2(8251):832-834; Berkner and Sharp (1983) Nucleic Acids Res. 11 (17):6003-6020; Graham (1984) EMBO J. 3:2917-2922; Bett et al. (1993) J. Virology 67:5911-5921; and Bett et al. (1994) Proc. Natl. Acad. Sci. USA 91:8802-8806. These references describe adenoviruses that have been genetically modified to produce replication-defective gene transfer vehicles. In such vehicles, the early adenovirus gene-products E1A and E1B are deleted and provided in trans by the packaging cell line 293 developed by Frank Graham (Graham et al. (1987) J. Gen. Virol. 36:59-72 and Graham (1977) J. Genetic Virology 68:937-940). The gene to be transduced is commonly inserted-into adenovirus in the deleted E1A and E1B region of the virus genome Bett et al. (1994), supra Adenovirus vectors as vehicles for efficient transduction of genes have been described by Stratford-Perricaudet (1990) Human Gene Therapy 1:2-256; Rosenfeld (1991) Science 252:431-434; Wang et al. (1991) Adv. Exp. Med. Biol. 309:61-66; Jaffe et al. (1992) Nat. Gen. 1:372-378; Quantin et al. (1992) Proc Natl. Acad. Sci. USA 89:2581-2584; Rosenfeld et al. (1992) Cell68:143-155; Stratford-Perricaudetet al. (1992) J. Clin. Invest. 90:626-630; Le Gal La Salle et al. (1993) Science 259:988-990; Mastrangeli et al. (1993) J. Clin. Invest. 91:225-234; Ragot et al. (1993) Nature 361:647-650; Hayaski et al. (1994) J. Biol. Chem. 269:23872-23875.

Other preferred vectors include the retroviral vectors described in Naldini, L. et al. ((1996) Science 272:263-267, incorporated herein by reference), as well as the vectors described in U.S. Pat. Nos. 6,428,953, 6,165,782, 6,013,516, and 5,994,136, all of which are incorporated herein by reference.

Another aspect of the invention pertains to host cells into which the nucleic acid molecules of the invention are introduced, e.g., an siRNA nucleic acid molecule within a vector (e.g., a recombinant adenoviral vector) or a modified adenoviral VA1 promoter nucleic acid molecule, which may or may not containing sequences which allow it to recombine into the host cell's genome. The terms ‘host cell’ and ‘recombinant host cell’ are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a vector containing an siRNA expression construct can be propagated and/or expressed in bacterial cells such as E. coli, insect cells, yeast or mammalian cells (such as Chinese hamster ovary cells (CHO), COS cells (e.g., COS7 cells), C6 glioma cells, HEK 293T cells, or neurons). Other suitable host cells are known to those skilled in the art. In a preferred embodiment, a host cell is a human cancer cell (e.g., a prostate cancer cell).

Vector DNA can be introduced into prokaryotic or eukaryotic cells via conventional transformation or transfection techniques. As used herein, the terms ‘transformation’ and ‘transfection’ are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an siRNA can be introduced on a separate vector. Cells stably transfected with the introduced nucleic acid can be identified by drug selection (e.g., cells that have incorporated the selectable marker gene will survive, while the other cells die).

In a most preferred embodiment, host cells containing the siRNAs of the invention are produced by infecting cells with a recombinant adenovirus containing the constructs.

This invention is further illustrated by the following examples which should not be construed as limiting. The contents of all references, patents, and published patent applications cited throughout this application, as well as the figures and the sequence listing, are incorporated herein by reference.

### EXAMPLES

### Example 1

### Enhanced Radiation and Chemotherapy-Mediated Cell Killing of Human Cancer Cells by Small Inhibitory RNA Silencing of DNA Repair Factors

Materials and Methods

Cell Culture

DU 145 and PC-3 cells were obtained from the American Type Culture Collection and maintained as adherent monolayer cultures in RPMI culture medium (Life Technologies, Inc., Carlsbad, Calif.), supplemented with 10% fetal bovine serum (Life Technologies). All cultures were grown at 37° C. in a humidified atmosphere of 5% carbon dioxide, fed every 5 days with complete medium, and subcultured when confluence was reached.

Plasmids

siRNAs were designed to target nucleotides 223-253 (G/C content=45%), 432-462 (G/C content=55%), and 597-627 (G/C content=42%) of the ATM mRNA (GenBank Accession No. U33841); nucleotides 134-164 (G/C content=48%), 388-418 (G/C content=48%), and 579-609 (G/C content=58%) of the ATR mRNA (GenBank Accession No. U76308); and nucleotides 196-226 (G/C content=58%), 585-616 (G/C content=42%), and 733-763 (G/C content=48%) of the DNA-PY_{cs} (GenBank Accession No. U47077) mRNA sequences with corresponding AUG translation initiation codons of nucleotides 190, 80, and 58, respectively.

The siRNA-encoding complementary single-stranded oligonucleotides, which hybridize to give BseRI- and BamHI-compatible overhangs, were designed using the RNAi OligoRetriever computer program, which is available on the Internet. The specific sequences used are shown in FIG. 14.

Oligonuceotides encoding siRNAs were ligated into the pSHAG-1 plasmid (Paddison, P. J. et al. (2002) Genes Dev. 16:948-958). Name designation of the resulting plasmids was pATM-1, -2, or -3; pAIR-1, -2, or -3; and pDNA-PK-1, -2, or -3 based on the target protein and region of mRNA downstream from the AUG codon (1 being the closest to the AUG codon). The pREV vector (Collis, S. J. et al. (2001) Nucleic Acids Res. 29:1534-1538), which encodes EGFP, was used for cotransfection studies and to assess transfection efficiencies.

Transfection of Cells

A total of 2×10⁵ cells was seeded into each well of a six-well tissue culture plate (Falcon, Bedford, Mass.). The next day (when the cells were 70-80% confluent), the culture medium was aspirated, and the cell monolayer was washed with prewarmed sterile PBS. Cells were transfected with the appropriate construct using LipofectaminePlus reagent (Life Technologies) according to the manufacturer's protocol. Green fluorescence of pREV-transfected cells was quantified at each time point by FACS analysis and used to ascertain transfection efficiencies for cells transiently transfected with siRNA encoding plasmids.

FACS Analysis

For each sample, 1×10⁴ cells were analyzed on a LSR flow cytometer (BD Biosciences, San Jose, Calif.) with an excitation wavelength of 488 nm and FITC collection wavelength using a band-pass filter at 530±15 nm. Dead cells were gated out of the samples by forward and side scatter. The level of EGFP fluorescence in live cells was determined using the Becton Dickinson CellQuest program. FACS sorting (≧10² fluorescence on a four-log scale) was carried out at the same excitation/emission wavelengths using a BD FACS Vantage SE (BD Biosciences).

Clonogenic Survival

At the appropriate time after transfection, cells were trypsinized and diluted to the appropriate cell density into 100-mm culture dishes to give ≧50 colonies/dish after irradiation and then irradiated at 0.78 Gy/min to the desired dose using a Gammacell 40 ¹³⁷cesium irradiator (Atomic Energy, Ottawa, Canada). For MMS clonogenic assays, at the appropriate time after transfection, cells were treated for 1 hour with MMS solubilzed in DMSO and diluted in serum-free media (SFM) or an equivalent percentage of DMSO/SFM as a control, washed twice in PBS, and trypsinized and plated as explained above. Ten days after radiation or drug treatment, colonies comprising ≧50 cells were counted after staining with 50% Crystal Violet (Sigma-Aldridge, St. Louis, Mo.). Cell survival was plotted as a function of dose and fitted using the linear quadratic model S=exp.(−αD−βD²), where S is the cell survival, D is the dose of radiation, and α and β are constants. DRFs, the factor by which the dose of radiation or drug can be reduced in the presence of the sensitizing agent to achieve the same level of cell killing in the absence of the sensitizing agent, were calculated as the dose required to give 10% cell survival (90% clonogenic cell killing) from fitted clonogenic survival curves for pSHAG-1−transfected÷siRNA-transfected cells. DRFs were calculated at 80 and 30% clonogenic cell killing for cells respectively treated with LY294002 and MMS, because the fitted survival curves did not reach 10% cell survival.

Immunoblots

Whole cell extracts were separated on 4-15% acrylamide gels (BioRad, Hercules, Calif.) using standard SDS-PAGE techniques. Antibodies for DNA-PK_{cs}, and β-actin were obtained from Oncogene Research Products (San Diego, Calif.), and Sigma Biochemicals (St. Louis, Mo.), respectively. A total of 2-20 μg of protein extracted from each transfected cell population was loaded onto each gel, electrophoresed at 100 V for 3 h at 4° C., and then transferred overnight at 50 mA, 4° C., onto polyvinylidene difluoride membranes (BioRad). Membranes were probed with primary and secondary antibodies at optimized concentrations, and protein expression was visualized using an enhanced chemiluminescence kit (Amersham-Pharmacia, Piscataway, N.J.). Membranes probed for ATM, ATR, and DNA-PKcs were reprobed for β-actin to normalize for loading and/or quantification errors and to allow comparisons of target protein expression to be made between transfected and untransfected populations. Protein expression was quantified using a Vesa-Doc gel documentation system (BioRad).

siRNA-mediated Down-Regulation of ATM, ATR and DNA-PK_{cs}

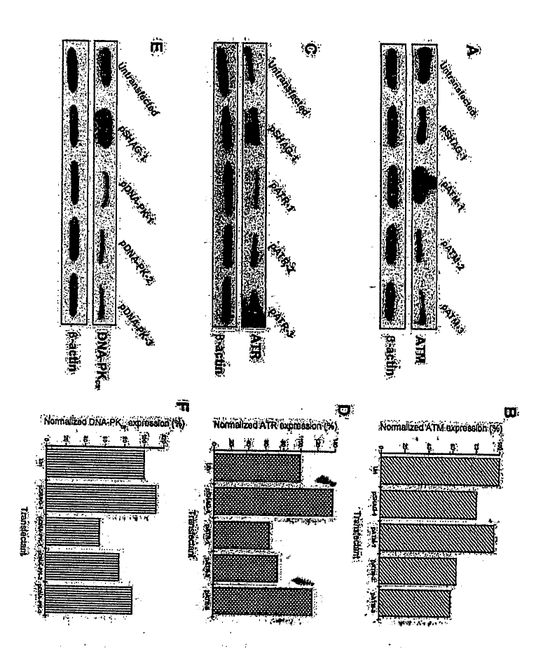

For each protein target, siRNA was designed complimentary to three different regions of the corresponding mRNA at increasing distance from the AUG translation initiation codon. Each siRNA was synthesized as complimentary oligonucleotides and cloned in the pSHAG-1 vector (Paddison, P. J. et al. (2002) Genes Dev. 16:948-958). The resulting constructs were then screened for their ability to down-regulate target protein expression. The human prostate cancer line DU 145 was transfected with each pSHAG-1/siRNA construct; protein extracts were obtained from 24-96-hours posttransfection, and Western blot analyses were performed for target protein and normalized to β-actin expression. We found that 48-hours post-transfection was the time at which greatest down-regulation (≦90% in transfected cells) was observed (FIG. 1). By 96-h posttransfection, target protein levels had risen back to levels comparable with empty vector-transfected and untransfected cells. For both ATR and DNA-PK_{cs}, the greatest down-regulation was seen with the siRNA targeting the region closest to the translation initiation sequence (pATR-1 and pDNA-PK-1, respectively), whereas the two regions further downstream in the mRNA sequence gave the highest amount of protein inhibition for ATM (pATM-2 and pATM-3).

siRNA Silencing of Repair Proteins Renders Human Prostate Tumor Cells Sensitive to DNA-Damaging Agents

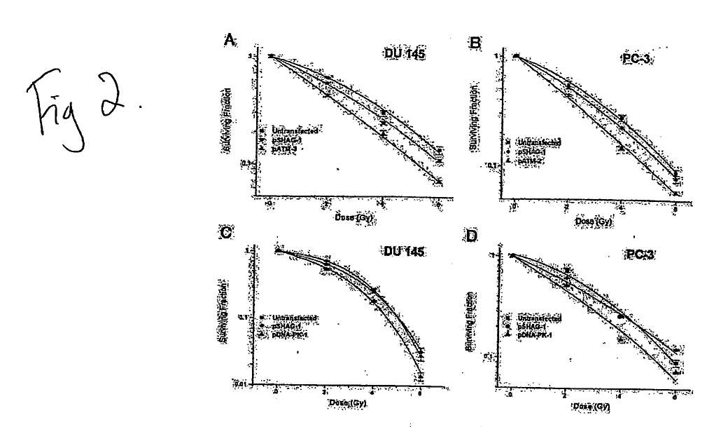

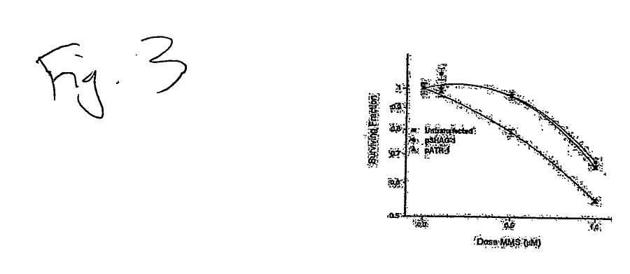

Previous studies have demonstrated that loss of function of ATM, ATR, or DNA-PK_{cs} results in increased cellular sensitivity to DNA-damaging agents. To ascertain if siRNA-mediated attenuation of expression of ATM, ATR, and DNA-PK_{cs} results in a subsequent sensitizing effect to such modalities, DU 145 and PC-3 human prostate cancer cells were transiently transfected with the ATM and DNA-P_{cs}-targeted, siRNA encoding plasmids that were shown to give the greatest inhibition of target protein expression. At the appropriate time post-transfection where protein levels were shown to be the lowest, the resulting heterogeneously transfected cultures were treated with ionizing radiation. Cellular sensitivity was ascertained by clonogenic survival assays (FIG. 2). siRNA-mediated inhibition of these DNA repair proteins conferred an increased sensitivity to ionizing radiation in siRNA transfected cell populations compared with untransfected or pSHAG-transfected cells. This increased radiosensitivity corresponded to DRFs of 1.1 and 1.21 for DNA-PK_{cs} silencing in DU 145 and PC-3 cells, respectively, and 1.16 and 1.14 for ATM silencing in DU 145 and PC-3 cells, respectively, with an increase in sensitivity of ˜1.5-1.8-fold noted at 6 Gy. In addition, DU 145 cells transfected with ATR-targeted, siRNA-encoding plasmids exhibited an increased sensitivity (DRF=1.38) to the alkylating agent MMS (FIG. 3). In a similar set of experiments, the expression of nontargeted siRNA in these cells failed to result in any evident radiation sensitization (DRF=0.99). In addition, transfection of cells with siRNA plasmids that effectively reduced ATR protein levels also failed to enhance radiosensitivity (DRF=0.98). Together, these data suggest that transfection and subsequent expression of plasmid-based siRNA does not, itself, result in an altered radiation response phenotype but actually requires specific targeting to produce such phenotypic alteration.

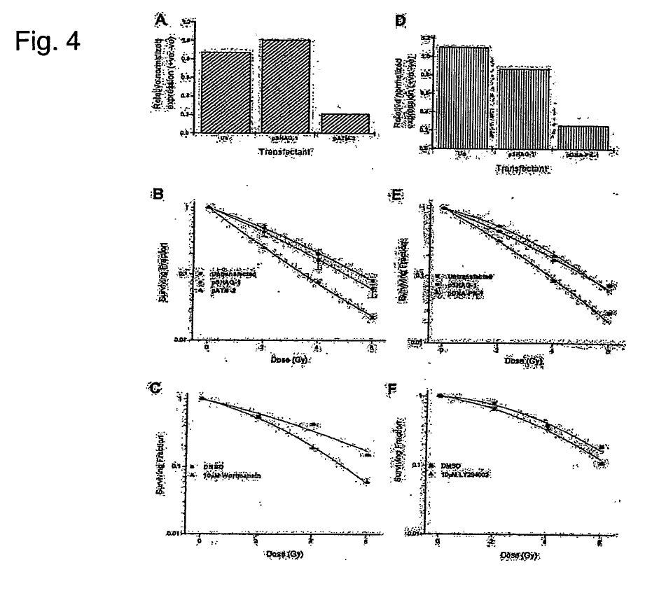

The biologically significant, but modest, degree of radiosensitization observed after transfection of ATM and DNA-PK_{cs} siRNA-encoding plasmids (FIG. 2) is a function of the heterogeneous populations resulting from transient transfection where the transfection efficiencies are 25-40%. To enrich the transfected population and demonstrate a more representative clonogenic survival of the transfected/siRNA-expressing cells, DU 145 cells were co-transfected with plasmids encoding siRNA-targeting ATM or DNA-PK_{cs} together with a plasmid EGFP and FACS-sorted, EGFP-expressing cells 48 hours posttransfection. Western blot analyses of protein extracts from FACS sorted cells transfected with siRNA-encoding plasmids confirmed that EGFP-expressing, FACS-sorted cells had vastly reduced target protein expression compared with those deemed untransfected attributable to a lack of EGFP expression (FIGS. 4A and D, respectively). Clonogenic survival assays demonstrated that the FACS-enriched ATM and DNA-PK_{cs} siRNA-transfected cells exhibited a substantial increased sensitivity to ionizing radiation (respective DRFs of 1.46 and 1.36, with an increase in sensitivity of ˜3-fold noted at 6 Gy) compared with cells transfected with just the EGFP-encoding plasmid or those transfected with pSHAG-1 (FIGS. 4B and E, respectively).

To highlight and further characterize the amount of radiosensitization caused by ATM and DNA-PKcs siRNA, the increase in radiosensitivity caused by the PI3k inhibitor Wortmannin, which inhibits ATM, ATR, and DNA-PKcs activity, and the specific DNA-PKcs competitive inhibitor LY294002 were assessed. DU 145 cells were treated with either DMSO, 10 μM Wortmannin, or 10 μM LY294002 one hour before and 24 hours after exposure to ionizing radiation, and cellular radiosensitivity was determined by clonogenic survival assays (FIGS. 4C and F). Treatment with Wortmannin and LY294002 led to an increased radiosensitivity (respective DRFs of 1.4 and 1.1) that was less than that caused by ATM or DNA-PK_{cs} siRNA (DRFs of 1.46 and 1.36; FIG. 4, B and E), thus demonstrating the efficacy of siRNA targeting specific DNA repair factors.

Discussion

Those siRNAs exhibiting the greatest inhibition of target protein expression were used to transfect cells before exposure to DNA-damaging agents and subsequently gave rise to a significant increase in sensitivity to ionizing radiation and alkylating agents compared with empty vector-transfected and untransfected control cells. This work demonstrates the first use of siRNA to augment radiation-mediated killing of human cancer cells and highlights the use of siRNA as an adjuvant gene therapy strategy to radiation and chemotherapy.

The radiation resistant prostate cancer cells DU 145 and PC-3 were transiently transfected with plasmids encoding siRNA and treated with commonly used classes of cancer therapeutics, namely ionizing radiation and alkylating agents (MMS). Cell survival was determined using clonogenic survival assays. Cells transfected with siRNA-encoding plasmids were rendered sensitive to ionizing radiation (via targeting of ATM and DNA-PKcs) and the alkylating agent MMS (ATR).

Initial screening of siRNA for their effectiveness showed that all three target proteins were down-regulated by 90% from 24- to 72-h post-transfection (FIG. 1), with protein levels being comparable with the levels seen in untransfected and vector-transfected cells at 96 h. These findings are consistent with previous data reporting the half-lives of these proteins to be in the region of 24-48 h (Lees-Miller, S. P. et al. (1996) J. Virol. 70:7471-7477; Fan, Z. et al. (2000) Cancer Gene Ther. 7:1307-1314).

The data presented herein demonstrate that siRNA-mediated inhibition in the expression of DNA repair proteins confers an increased sensitivity to therapeutically relevant DNA-damaging agents (FIGS. 2 and 3). For ATM and DNA-PK_{cs}, siRNA-mediated attenuation of protein expression was manifested as an increased radiosensitivity for both heterogeneously transfected cells and FACS-enriched cells (respective DRFs of 1.15 and 1.4, with a 1.5-1.8- and 3-fold increase in radiosensitivity at 6 Gy in heterogeneously and transfected cells, respectively; FIGS. 2 and 4). In addition, siRNA-mediated inhibition of ATR expression resulted in an increased sensitivity to the alkylating agent MMS (FIG. 3). Although DU 145 cells are somewhat resistant to alkylating agents because of a mutation in the mismatch repair protein hMLH1 (Chen, Y. et al. (2001) Cancer Res. 61:4112-4121; Yeh, C. C. et al. (2001) Biochem. Biophys. Res. Commun. 285:409-413), transfection of only 50% of cells with an ATR siRNA-encoding plasmid gave rise to an overall decreased cell survival (DRF of 1.38) after treatment with MMS (FIG. 3).

Although the observed increased radiation-mediated cell killing caused by DNA-PK_{cs} and ATM siRNA appears modest (average DRFs of 1.15; FIG. 2), it has to be emphasized that the survival curves shown represent a population of cells where only a minority (25-40%) are transfected and, thus, also have depleted levels of each target protein. To address this problem, DU 145 cells were cotransfected with the siRNA-encoding plasmid and a plasmid encoding EGFP, which were used to enrich the transfected population via FACS sorting. Western blot analysis showed that the FACS-sorted cells had a 80% reduction in both ATM and DNA-PK_{cs} expression (FIG. 4, A and D), which resulted in biologically significant DRFs of 1.46 and 1.36, with a 3-fold increased sensitivity noted at 6 Gy (Ref. 19; FIG. 4, B and E). Furthermore, the radiosensitivity exhibited by the enriched cells was greater than that seen in cells treated with the PI3k inhibitor Wortmannin or specific DNA-PKcs competitive inhibitor LY294002 (respective DRFs of 1.4 and 1.1; FIG. 4, C and F). The use of LY294002 before and after exposure to radiation highlights the increase in radiosensitivity that can be achieved by inhibition of DNA-PKcs activity in DU 145 cells. Moreover, this approach also results in a slightly greater radiosensitization than that seen in normal human fibroblasts transfected with doubled-stranded siRNA targeting DNA-PK_{cs} (Peng, Y. et al. (2002) Cancer Res. 62:6400-6404).

### Example 2

### Construction of a Synthetic VA1 Promoter to Drive siRNA Expression

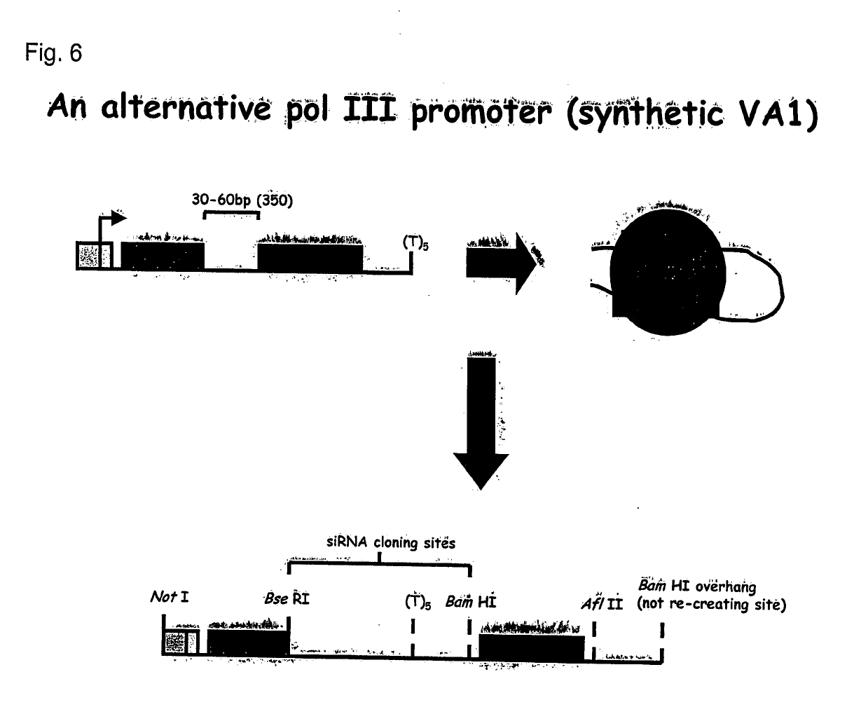

This example describes the construction of a synthetic, modified VA1 promoter for use in expressing siRNA expression when contained in a adenovirus. A schematic of the natural VA1 adenoviral promoter is shown in FIG. 6. The sequence of the natural VA1 promoter is shown in FIG. 24 (SEQ ID NO:37). To produce the modified VA1 promoter, cloning sites for insertion of the siRNA-encoding nucleic acid:molecules were added between the A-Box and the B-Box. The natural termination sequence was also moved from 5′ of the B-Box to in between the A-Box and the B-Box.

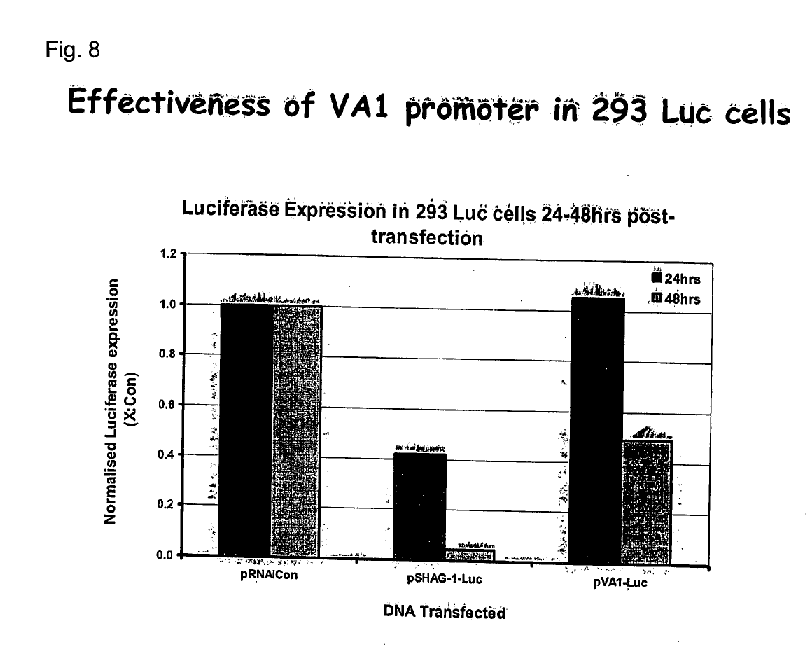

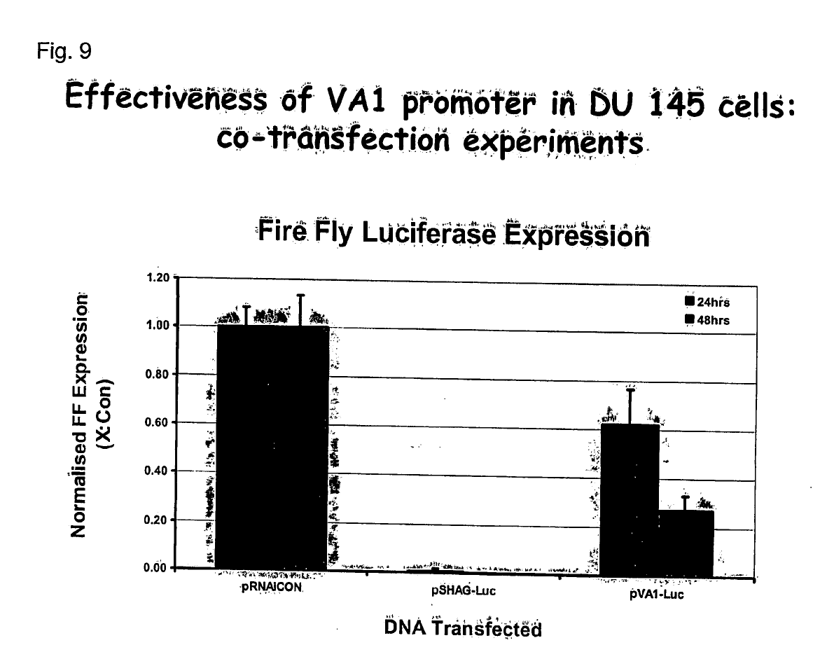

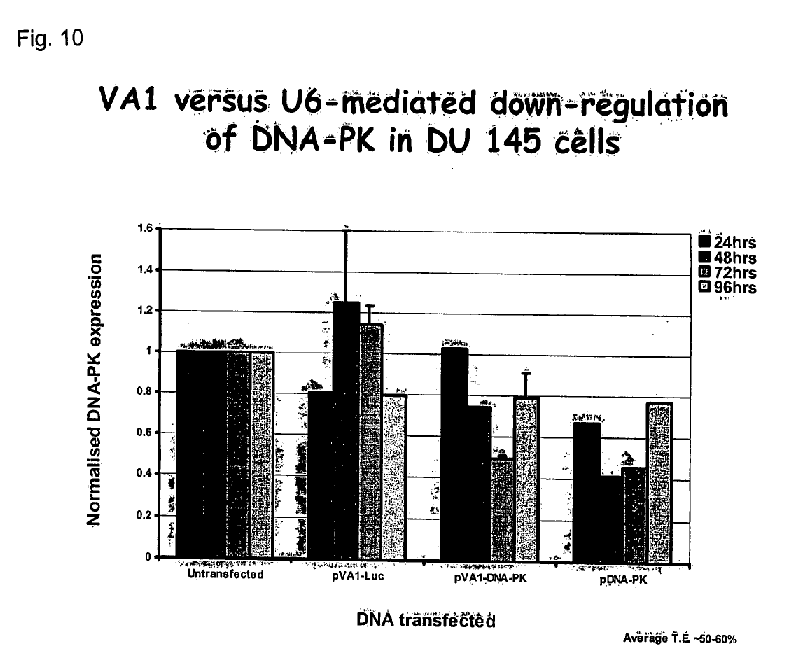

To test the effectiveness of the modified VA1 promoter, a nucleic acid molecule encoding an siRNA targeting the luciferase mRNA was inserted into the cloning sites in the modified promoter (FIG. 25), and the construct was transfected into PC-3 Luc cells, which constitutively express luciferase. As shown in FIG. 8, the modified VA1 promoter was effective as a promoter in downregulating luciferase expression. The modified VA1 promoter was also effective using 293 Luc cells (FIG. 9), which also constitutively express luciferase. FIG. 10 shows the effectiveness of the modified VA1 promoter in downregulating luciferase expression in DU 145 cells when the luciferase is expressed from a cotransfected plasmid.

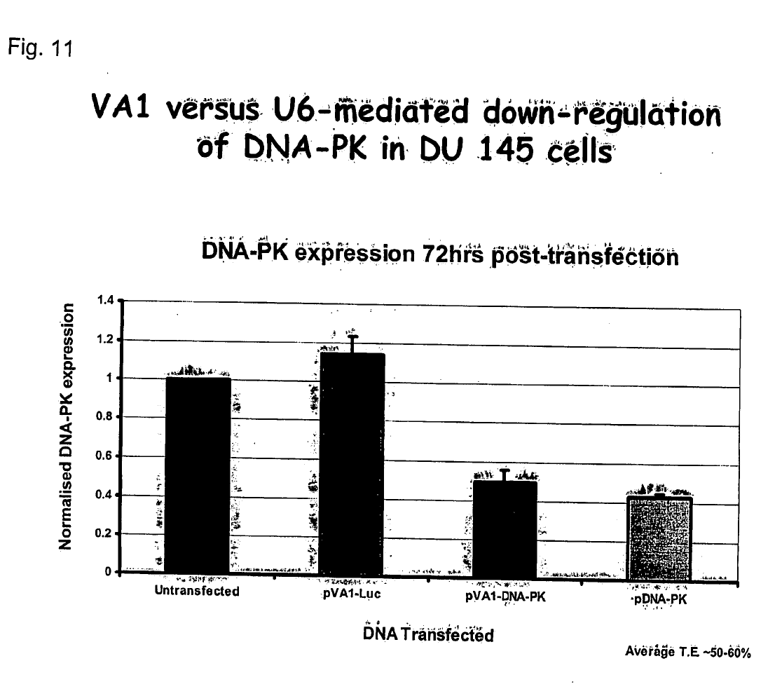

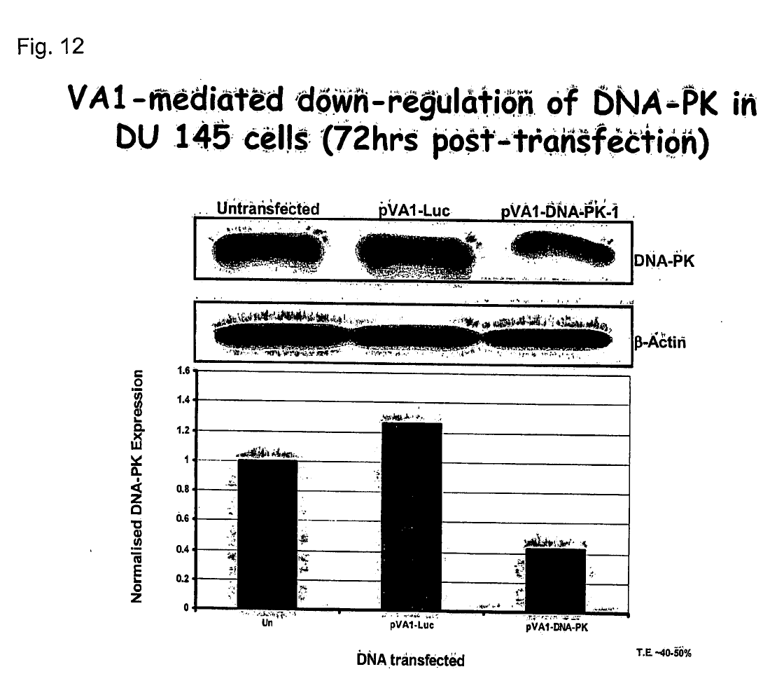

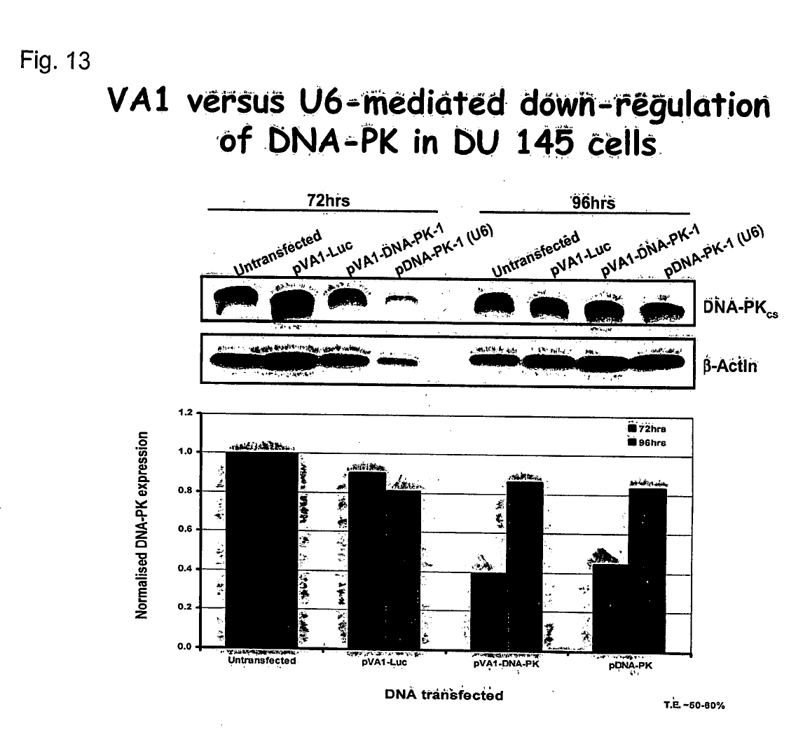

When used to express an siRNA targeting DNA-PK in DU 145 cells, the VA1 promoter is as effective at 72 hours as the U6 promoter (FIGS. 11 and 12). FIGS. 13 and 14 show that the reduction in DNA-PK is specific to DNA-PK, and not to overall protein synthesis reduction.

Equivalents

Those skilled in the art will recognize, or be able to ascertain using no more than routine experimentation, many equivalents to the specific embodiments of the invention described herein. Such equivalents are intended to be encompassed by the following claims.

1. A method of killing a tumor cell comprising contacting the cell with at least one small inhibitory RNA (siRNA) specific for a DNA repair protein and at least one DNA-damaging agent.

2. The method of claim 1, wherein the DNA repair protein is selected from the group consisting of ATM, ATR, and DNA-PK_{cs}.

3. The method of claim 1, wherein the siRNA is encoded by a nucleic acid sequence selected from the group consisting of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

4-22. (canceled)

23. A method of treating a subject having cancer comprising administering to the subject a therapeutically effective amount of at least one small inhibitory RNA (siRNA) specific for a DNA repair protein and a therapeutically effective amount of at least one DNA-damaging agent.

24. The method of claim 23, wherein the DNA repair protein is selected from the group consisting of ATM, ATR, and DNA-PK_{cs}.

25. The method of claim 23, wherein the siRNA is encoded by a nucleic acid sequence selected from the group consisting of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.

26-49. (canceled)

50. An isolated nucleic acid molecule comprising a modified adenoviral promoter, wherein the modified adenoviral promoter comprises, in sequence, an adenoviral VA1 A-Box, at least one restriction enzyme recognition site, at least one adenoviral VA1 termination sequence, and at least one adenoviral VA1 B-box.

51. A isolated nucleic acid molecule comprising a nucleic acid sequence selected from the group consisting of SEQ ID NO:1, 2, 4, 5, 7, 8, 10, 11, 13, 14, 16, 17, 19, 20, 22, 23, 25, 26, 28, 29, 30, 31, 32, 33, 34, 35, or 36.
